(19) United States
(12) Patent Application Publication
(10) Pub. No.: US 2023/0059978 A1
(43) Pub. Date: Feb. 23, 2023

(54) LIVE CELL CONSTRUCTS FOR BIOSYNTHETIC MILK PRODUCTION AND RELATED PRODUCTS AND METHODS

(71) Applicants: Shayne Guiliano, Hillsborough, NC (US); Leila Strickland, Hillsborough, NC (US)

(72) Inventors: Shayne Guiliano, Hillsborough, NC (US); Leila Strickland, Hillsborough, NC (US)

(21) Appl. No.: 17/791,106

(22) PCT Filed: Jan. 8, 2021
(86) PCT No.: PCT/US2021/012676

§ 371 Date: Jul. 6, 2022

Related U.S. Application Data
(60) Provisional application No. 62/958,407, filed on Jan. 8, 2020.

(51) Int. Cl. C12N 5/071

(52) U.S. Cl. CPC C12N 5/0631; C12N 2533/52; C12N 2533/54; C12N 2533/72; C12N 2533/74; C12N 2533/80

(57) ABSTRACT

This invention relates to live cell constructs for producing milk in culture and compositions comprising a milk product produced by the live cell contracts, as well as methods for making a live cell construct for producing milk in culture, methods of producing milk in culture, and methods of producing a modified primary mammary epithelial cell or an immortalized mammary epithelial cell for use in a live cell construct and other methods of the present invention.

### FIELD OF THE INVENTION

This invention relates to live cell constructs and methods of using same for in vitro and/or ex vivo production of milk from cultured mammary cells.

### BACKGROUND OF THE INVENTION

Milk is a staple of the human diet, both during infancy and throughout life. The American Academy of Pediatrics and World Health Organization recommend that infants be exclusively breastfed for the first 6 months of life, and consumption of dairy beyond infancy is a mainstay of human nutrition, representing a 700 billion dollar industry worldwide. However, lactation is a physiologically demanding and metabolically intensive process that can present biological and practical challenges for breastfeeding mothers, and milk production is associated with environmental, social, and animal welfare impacts in agricultural contexts.

The possibility of using mammalian cell culture to produce food has gained increasing interest in recent years, with the development of several successful prototypes of meat and sea food products from cultured muscle and fat cells (Stephens et al. 2018 Trends Food Sci Technol. 78:155-166). Additionally, efforts are underway to commercialize the production of egg and milk proteins using microbial expression systems. However, this fermentation-based process relies on the genetically engineered expression and purification of individual components and is unable to reproduce the full molecular profile of milk or dairy.

The present invention overcomes shortcomings in the art by providing live cell constructs and methods using the same for in vitro and/or ex vivo production of milk from cultured mammary cells.

### SUMMARY OF THE INVENTION

The present invention is based, in part, on the development of live cell constructs comprising mammary cells that compartmentalize feeding of the cells and secretion of milk.

Thus, one aspect of the invention relates to a live cell construct comprising, a scaffold having a top surface and a bottom surface; and a continuous monolayer of (a) live primary mammary epithelial cells, (b) a mixed population of live primary mammary epithelial cells, mammary myoepithelial cells and mammary progenitor cells, and/or (c) live immortalized mammary epithelial cells on the top surface of the scaffold, the continuous monolayer of (a) live primary mammary epithelial cells, (b) mixed population of live primary mammary epithelial cells mammary myoepithelial cells and mammary progenitor cells, and/or (c) immortalized mammary epithelial cells having an apical surface and a basal surface (e.g., the cells form a polarized and confluent cell monolayer), wherein the construct comprises an apical compartment above and adjacent to the apical surface of the continuous monolayer of the (a) live primary mammary epithelial cells, the (b) mixed population of live primary mammary epithelial cells, mammary myoepithelial cell and mammary progenitor cells, and/or the (c) immortalized mammary epithelial cells and a basal compartment below and adjacent to the bottom surface of the scaffold.

Another aspect of the invention provides a method of producing milk in culture, the method comprising culturing the live cell construct of the present invention, thereby producing milk in culture.

An additional aspect of the invention provides a method of making a live cell construct for producing milk in culture, the method comprising (a) isolating primary mammary epithelial cells, myoepithelial cells and/or mammary progenitor cells from mammary explants from mammary tissue, to produce isolated mammary epithelial cells, myoepithelial cells and mammary progenitor cells; (b) culturing the isolated primary mammary epithelial cells, myoepithelial cells and mammary progenitor cells to produce a mixed population of primary mammary epithelial cells, mammary myoepithelial cells and mammary progenitor cells; (c) cultivating the mixed population of (b) on a scaffold, the scaffold having an upper surface and lower surface, to produce a polarized, continuous (i.e., confluent) monolayer of primary mammary epithelial cells, myoepithelial cells and mammary progenitor cells of the mixed population on the upper surface of the scaffold, wherein the polarized, continuous monolayer comprises an apical surface and a basal surface, thereby producing a live cell construct for producing milk in culture.

A further aspect of the present invention relates to a method of making a live cell construct for producing milk in culture, the method comprising: a) isolating primary mammary epithelial cells, myoepithelial cells, and/or mammary progenitor cells from mammary explants from mammary tissue (e.g., breast, udder, teat tissue), to produce isolated mammary epithelial cells, myoepithelial cells, and/or mammary progenitor cells; (b) culturing the isolated primary mammary epithelial cells, myoepithelial cells, and/or mammary progenitor cells to produce a mixed population of primary mammary epithelial cells, mammary myoepithelial cells and mammary progenitor cells; (c) sorting the mixed population of primary mammary epithelial cells, myoepithelial cells, and/or mammary progenitor cells to produce a population of primary mammary epithelial cells; and (d) cultivating the population of primary mammary epithelial cells on a scaffold, the scaffold having an upper surface and lower surface, to produce a polarized, continuous (i.e., confluent) monolayer of primary mammary epithelial cells on the upper surface of the scaffold, wherein the polarized, continuous monolayer comprises an apical surface and a basal surface, thereby producing a live cell construct for producing milk in culture.

Another aspect of the present invention relates to a method of making a live cell construct for producing milk in culture, the method comprising (a) culturing immortalized mammary epithelial cells to produce increased numbers of immortalized mammary epithelial cells; (b) cultivating the immortalized mammary epithelial cells of (a) on a scaffold, the scaffold having an upper surface and lower surface, to produce a polarized, continuous (i.e., confluent) monolayer of immortalized mammary epithelial cells on the upper surface of the scaffold, wherein the polarized, continuous monolayer comprises an apical surface and a basal surface, thereby producing a live cell construct for producing milk in culture.

Another aspect of the present invention relates a method of producing milk in culture comprising, culturing a live cell construct comprising (a) a scaffold comprising an upper surface and a lower surface and a continuous (i.e., confluent) polarized monolayer of live primary mammary epithelial cells, a continuous polarized monolayer of a mixed population of live primary mammary epithelial cells, mammary myoepithelial cells and mammary progenitor cells, and/or a continuous polarized monolayer of live immortalized mammary epithelial cells having an apical surface and a basal surface, wherein the continuous polarized monolayer of live primary mammary epithelial cells, the continuous polarized monolayer of the mixed population of live primary mammary epithelial cells, mammary myoepithelial cells and mammary progenitor cells and/or the continuous polarized monolayer of live immortalized mammary epithelial cells are located on the upper surface of scaffold, (b) a basal compartment and an apical compartment, wherein the lower surface of the scaffold is adjacent to the basal compartment and the apical surface of the monolayer of live primary mammary epithelial cells, the monolayer of the mixed population of live primary mammary epithelial cells, mammary myoepithelial cells and mammary progenitor cells, and/or the monolayer of live immortalized mammary epithelial cells is adjacent to the apical compartment, wherein the monolayer of live primary epithelial mammary cells, the live primary epithelial mammary cells of the monolayer of the mixed population of live primary mammary epithelial cells, mammary myoepithelial cells and mammary progenitor cells, or the monolayer of immortalized mammary epithelial cells excretes milk through its apical surface into the apical compartment, thereby producing milk in culture.

A further aspect of the present invention relates to a method of producing a modified primary mammary epithelial cell or a immortalized mammary epithelial cell, wherein the method comprises introducing into the cell: (a) a polynucleotide encoding a prolactin receptor comprising a modified intracellular signaling domain, optionally wherein the prolactin receptor comprises a truncation wherein position 154 of exon 10 has been spliced to the 3′ sequence of exon 11; (b) a polynucleotide encoding a chimeric prolactin receptor that binds to a ligand, which is capable of activating milk synthesis in the absence of prolactin; (c) a polynucleotide encoding a constitutively or conditionally active prolactin receptor protein, optionally wherein the polynucleotide encodes a constitutively active human prolactin receptor protein comprising a deletion of amino acids 9 through 187; (d) a polynucleotide encoding a modified (recombinant) effector of a prolactin protein comprising (i) a JAK2 tyrosine kinase domain fused to a STATS tyrosine kinase domain; and/or (ii) a prolactin receptor intracellular domain fused to a JAK2 tyrosine kinase domain; (e) a loss of function mutation into a circadian related gene PER2 (period circadian protein homolog 2); and/or (f) a polynucleotide encoding one or more glucose transporter genes GLUT1 and/or GLUT12, thereby increasing the rate of nutrient uptake at the basal surface of a monolayer of cells of the modified primary mammary epithelial cell or immortalized mammary epithelial cell.

A further aspect of the present invention relates to compositions comprising a biosynthetic milk product produced by a live cell construct described herein and compositions comprising a biosynthetic milk product produced by a method described herein.

The present invention is also based, in part, on the successful production of a biosynthetic human milk product from primary human mammary epithelial cells (HUMECs) cultured in a hollow fiber bioreactor.

Thus, a further aspect of the invention relates to a live cell construct comprising lactating primary human mammary epithelial cells (HMECs) forming a continuous monolayer on a plurality of hollow capillary tubes arranged in a parallel array within a tubular cartridge defining an intracapillary (IC) space and an extracapillary (EC) space, each hollow capillary tube constucted of a semi-permeable membrane defining an internal surface adjacent to the IC space and an external surface adjacent to the EC space, wherein the external suface of each hollow capillary tube is coated with a mixture of collagen IV and laminin I and the HUMEC monolayer is in contact with the coated surface; and wherein a cell growth medium supplemented with prolactin fills the IC space. In embodiments, the semi-permeable membrane is fabricated from polyvinylidene difluoride (PVDF) or polysulfone and/or the semi-permeable membrane has a molecular weight cut-off (MWCO) between 5-80 kilodaltons (kDa).

Another aspect of the present invention relates to compositions comprising a biosynthetic human milk product produced by a live cell construct comprising a plurality of hollow capillary tubes.

In a further aspect, the invention relates to a biosynthetic human milk composition comprising a lipid component, a protein component, and a carbohydrate component, wherein the lipid, protein, and carbohydrate components each consist of human lipids, human proteins or peptides, and human carbohydrates, and wherein the composition is free of pathogens, cytotoxins, and genetically modified or engineered molecules. In this context, the reference to “human” components means lipids, proteins, and carbohydrates produced by human cells and naturally occuring in humans. In an aspect, the composition is free of pathogens including bacteria, viruses, and fungi. In an aspect, the composition is not pasteurized. In an aspect, the lipid component comprises 1-5% of the composition; the protein component comprises 0.5-1% of the composition; and the carbohydrate component comprises 6-8% of the composition. In an aspect, the lipid component comprises palmitic acid, oleic acid, and one or more bioactive lipid mediators of fatty acids. In an aspect, the one or more bioactive lipid mediators of fatty acids is an anti-inflammatory compound. In an aspect, the one or more bioactive lipid mediators of fatty acids is selected from the group consisting of epoxyoctadecenoic acid (EpOME); epoxyeicosatrienoic acid (EpETrE); epoxyeicosatetraenoic acid (EpETE); epoxydocosapentaenoic acid (EpDPE); dihydroxyoctadecenoic acid (DiHOME); dihydroxyeicosatrienoic acid (DiHETrE); dihydroxyeicosatetraenoic acid (DiHETE); hydroxyoctadecadienoic acid (HODE); hydroxyeicosatrienoicacid (HETrE); hydroxyeicosatetraenoic acid (HETE); hydroxyoctadecatrienoic acid (HOTrE); hydroxyeicosapentaenoic acid (HEPE); hydroxydocosahexaenoic acid (HdoHE); and leukotriene. In an aspect, the protein component comprises one or more proteins or peptides selected from the group consisting of alpha-lactalbumin, bile salt-activated lipase (BSAL), butyrophilin, casein, fatty acid synthase, insulin, lactadherin, lactoferrin, lactotransferrin, lysozyme, mucin-1, osteopontin, perilipin-2, serum albumin, and xanthine dehydrogenase/oxidase. In an aspect, the protein component comprises BSAL, lysozyme, and lactoferrin. In an aspect, the carbohydrate component comprises one or more of lactose, 2′ fucosyl lactose, myo-inositol, lacto-N-neotetraose (LNnT), 6′-sialyllactose, sialyl-lacto-N-tetraose, lacto-N-fucopentaose (LNFP) I, lacto-N-fucopentaose (LNFP) II, and disialyl-lacto-N-tetraose.

In a further aspect, the invention relates to methods for making a biosynthetic milk product, the method comprising expanding a population of human mammary epithelial cells (HUMECs) in a growth medium on a substrate comprising collagen IV; dislodging the expanded population of HUMECs from the substrate and seeding the dislodged HUMECs into a hollow fiber bioreactor containing capillaries pre-coated with a mixture of collagen IV and laminin I; culturing the HUMECs for a period of time until the HUMECs have reached confluence; and stimulating production of the biosynthetic milk product by contacting the HUMECs with prolactin using a method comprising contacting the cells with 100 ng/ml prolactin for a period of time followed by contacting the cells with 200 ng/ml prolactin for a second period of time. In an aspect, the HUMECs are selected from primary cells, primary immortalized cells, or recombinant cells. In an aspect, the method further comprises a step of preparing the bioreactor prior to seeding the HUMECs, wherein preparing the bioreactor comprises creating a negative pressure within the bioreactor and applying a 1:1 mixture of collagen IV and laminin I in phosphate buffered saline (PBS) to the hollow fibers. In an aspect, applying the mixture of collagen IV and laminin I is accomplished using a syringe inserted into a port of the bioreactor.

These and other aspects of the invention are set forth in more detail in the description of the invention below.

### BRIEF DESCRIPTION OF THE DRAWINGS

FIG. 1 shows an example of the collection of milk for nutritional use from mammary epithelial cells grown as a confluent monolayer in a compartmentalizing culture apparatus in which either fresh or recycled media is provided to the basal compartment and milk is collected from the apical compartment. TEER, transepithelial electrical resistance.

FIG. 2A-B panel A shows an example of polarized absorption of nutrients and secretion of milk across a confluent monolayer of mammary epithelial cells anchored to a scaffold at the basal surface; panel B shows an example micropatterned scaffold provides increased surface area for the compartmentalized absorption of nutrients and secretion of milk by a confluent monolayer of mammary epithelial cells.

FIG. 3A-C shows three views of a hollow fiber bioreactor comprising a bundle of capillary tubes (A), each capillary tube having an external and an internal surface, each surface defining a first internal compartment (intracapillary space, or IC) and a second external compartment (extracapillary space, or EC). The mammary epithelial cells may form a confluent monolayer either on the external surface (B) or on the internal surface (C) of the capillaries, providing directional and compartmentalized absorption of nutrients and secretion of milk.

FIG. 4 Glucose utilization (mg/day) by HUMECS cultured in a hollow fiber bioreactor over time post-seeding of the cells in the bioreactor as described in Example 2. Arrows indicate prolactin addition at day 11 (100 ng/ml), day 26 (200 ng/ml), and day 32 (100 ng/ml).

FIG. 5 Prolactin stimulation of HMECS cultured in a hollow fiber bioreactor as described in Example 2 results in dramatic increase of secreted protein. Chart shows prolactin addition (either 100 or 200 ng/ml) over time (days) following seeding of the bioreactor and total secreted protein over the same time period.

FIG. 6A-C Lactose (A) and 2′ fucosyl lactose (B) production (micromolar, uM) over time (days) by HMECs cultured in a hollow fiber bioreactor as described in Example 2. Charts also show time and amount of prolactin addition, 100 ng/ml starting at day 11; 200 ng/ml starting at day 26; and 100 ng/ml starting at day 32, as indicated by arrows. Panel C shows NMR spectra of some characteristic peaks for lactose and 2′ fucosyl lactose in the HMEC culture media (from bottom line 1); ECS harvest (line 2); reservoir (line 3); and human milk (line 4). The first three spectra are on the same scale; the human milk spectra has been reduced for presentation.

FIG. 7 Casein production over time by HMECs cultured in a hollow fiber bioreactor as described in Example 2. From left, lane 1 contains the molecular weight marker; lane 2 human milk; lanes 3-7 contain protein isolated from ECM harvest on days 22, 25, 26, 27, and 29 post-seeding of the bioreactor.

FIG. 8 Image of Coomassie-stained SDS-PAGE gel showing proteins produced by HMECs cultured in a hollow fiber bioreactor as described in Example 2 compared to proteins present in human milk. From left, lanes 1-4 and lanes 5-8 contain protein isolated from reservoir and ECM harvest, respectively, on days 31, 32, 33, and 36 post-seeding of the bioreactor.

FIG. 9 Protein harvest from HUMECS cultured in a hollow fiber bioreactor as described in Example 2.

FIG. 10 HPLC chromatogram of a sample of the biosynthetic milk product produced in Example 2. Graph is annotated to show peaks corresponding to some important milk proteins. Full sample spectrum is shown in light grey. Darker lines show the contribution from isolated proteins.

### DETAILED DESCRIPTION OF THE INVENTION

The present invention is explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which do not depart from the instant invention. Hence, the following specification is intended to illustrate some particular embodiments of the invention, and not to exhaustively specify all permutations, combinations and variations thereof.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

Nucleotide sequences are presented herein by single strand only, in the 5′ to 3′ direction, from left to right, unless specifically indicated otherwise. Nucleotides and amino acids are represented herein in the manner recommended by the IUPAC-IUB Biochemical Nomenclature Commission, or (for amino acids) by either the one-letter code, or the three letter code, both in accordance with 37 C.F.R. § 1.822 and established usage.

Except as otherwise indicated, standard methods known to those skilled in the art may be used for production of recombinant and synthetic polypeptides, antibodies or antigen-binding fragments thereof, manipulation of nucleic acid sequences, production of transformed cells, the construction of viral vector constructs, and transiently and stably transfected packaging cells. Such techniques are known to those skilled in the art. See, e.g., Sambrook et al., Molecular Cloning: A Laboratory Manual 2nd Ed. (Cold Spring Harbor, N.Y., 1989); F. M. Ausubel et al. Current Protocols In Molecular Biology (Green Publishing Associates, Inc. and John Wiley & Sons, Inc., New York).

All publications, patent applications, patents, nucleotide sequences, amino acid sequences and other references mentioned herein are incorporated by reference in their entireties for the teachings relevant to the sentence and/or paragraph in which the reference is presented.

### Definitions

As used in the description of the invention and the appended claims, the singular forms “a,” “an” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

Moreover, the present invention also contemplates that in some embodiments of the invention, any feature or combination of features set forth herein can be excluded or omitted.

Furthermore, the term “about,” as used herein when referring to a measurable value such as an amount of a compound or agent of this invention, dose, time, temperature, and the like, is meant to encompass variations of ±10%, ±5%, ±1%, ±0.5%, or even ±0.1% of the specified amount.

As used herein, the transitional phrase “consisting essentially of” is to be interpreted as encompassing the recited materials or steps and those that do not materially affect the basic and novel characteristic(s) of the claimed invention. Thus, the term “consisting essentially of” as used herein should not be interpreted as equivalent to “comprising.”

As used herein, the term “polypeptide” encompasses both peptides and proteins, unless indicated otherwise. The term “protein component” in the context of the biosynthetic human milk product described herein, encompasses peptides, polypeptides, and proteins.

The term “materially altered” (or grammatical equivalents, e.g., “modified”) as applied to polynucleotides and/or polypeptides of the invention, refers to a polynucleotide and/or polypeptide that is substantially modified from its native form in composition and/or genomic locus by deliberate human intervention

As used herein, by “isolate” (or grammatical equivalents, e.g., “extract”) a product, it is meant that the product is at least partially separated from at least some of the other components in the starting material.

By “substantially retain” a property, it is meant that at least about 75%, 85%, 90%, 95%, 97%, 98%, 99% or 100% of the property (e.g., activity or other measurable characteristic) is retained.

The term “polarized” as used herein in reference to cells and/or monolayers of cells refers to a spatial status of the cell wherein there are two distinct surfaces of the cell, e.g., an apical surface and a basal surface, which may be different (e.g., may comprise different surface and/or transmembrane receptors and/or other structures). Individual polarized cells in a continuous monolayer may have similarly-oriented apical surfaces and basal surfaces, and may have communicative structures between individual cells (e.g., tight junctions) to allow cross communication between individual cells and to create separation (e.g., compartmentalization) of the apical compartment (e.g., the lumen above and adjacent to the apical surface) and basal compartment (e.g., the lumen below and adjacent to the basal surface).

The term “lactogenic” as used herein refers to the ability to stimulate production and/or secretion of milk. A lactogenic product may be a gene, protein (e.g., prolactin), or other natural and/or synthetic product. A culture medium comprising lactogenic properties (e.g., comprising prolactin, thereby stimulating production of milk by cells in contact with the culture medium) may be referred to as a “lactogenic culture medium.”

As used herein, the term “food grade” refers to materials considered non-toxic and safe for consumption (e.g., human and/or other animal consumption), e.g., as regulated by standards set by the U.S. Food and Drug Administration.

As used herein, the term “genetically modified or engineered molecules” encompasses molecules produced by recombinant technology.

As used herein, the term “biosynthetic” in the context of “biosynthetic milk” refers to a milk product or composition secreted by cells cultured in vitro, and excludes milk products or compositions containing milk produced by a mammal in vivo, including human donor milk and human mother's milk.

### Live Cell Constructs

The present invention relates to live cell constructs, methods of making the same, and methods of using the same for in vitro and/or ex vivo production of milk from cultured mammary cells. Milk is a complex macromolecular secretion composed of proteins, lipids, and carbohydrates produced by epithelial cells that line the internal compartment of the mammary gland. Mammary epithelial cells in culture have been previously demonstrated to display organization and behavior similar to that observed in vivo (Arevalo et al. 2016 Am J Physiol Cell Physiol. 310(5):C348-356; Chen et al. 2019 Curr Protoc Cell Biol. 82(1):e65). In particular, when grown on an appropriate extracellular matrix and stimulated with prolactin, cultured mammary epithelial cells organize into polarized structures and secrete milk components (Blatchford et al. 1999 Animal Cell Technology: Basic & Applied Aspects 10:141-145). However, as previous studies have been focused on basic and biomedical research, nutritional applications of in vitro milk production remain unexplored and no attempt has been made to collect the milk separately from the medium in which the cells are grown.

Thus, one aspect of the invention relates to a live cell construct comprising, a scaffold having a top surface and a bottom surface; and a continuous monolayer of (a) live primary mammary epithelial cells, (b) a mixed population of live primary mammary epithelial cells, mammary myoepithelial cells and mammary progenitor cells, and/or (c) live immortalized mammary epithelial cells on the top surface of the scaffold, the continuous monolayer of (a) live primary mammary epithelial cells, (b) mixed population of live primary mammary epithelial cells mammary myoepithelial cells and mammary progenitor cells, and/or (c) immortalized mammary epithelial cells having an apical surface and a basal surface (e.g., the cells form a polarized and confluent cell monolayer), wherein the construct comprises an apical compartment above and adjacent to the apical surface of the continuous monolayer of the (a) live primary mammary epithelial cells, the (b) mixed population of live primary mammary epithelial cells, mammary myoepithelial cell and mammary progenitor cells, and/or the (c) immortalized mammary epithelial cells and a basal compartment below and adjacent to the bottom surface of the scaffold.

A live primary culture of mammary gland tissue may comprise milk-producing mammary epithelial cells, contractile myoepithelial cells, and/or progenitor cells that can give rise to both mammary epithelial and mammary contractile myoepithelial cells. Mammary epithelial cells are the only cells that produce milk. The live primary mammary epithelial cells, the mixed population of live primary mammary epithelial cells, mammary myoepithelial cells and mammary progenitor cells, and/or the immortalized mammary epithelial cells may be from any mammal, e.g., a primate (e.g., chimpanzee, orangutan, gorilla, monkey (e.g., Old World, New World), lemur, human), a dog, a cat, a rabbit, a mouse, a rat, a horse, a cow, a goat, a sheep, an ox (e.g., Bos spp.), a pig, a deer, a musk deer, a bovid, a whale, a dolphin, a hippopotamus, an elephant, a rhinoceros, a giraffe, a zebra, a lion, a cheetah, a tiger, a panda, a red panda, and an otter. In some embodiments, the live primary mammary epithelial cells, the mixed population of live primary mammary epithelial cells, mammary myoepithelial cells and mammary progenitor cells, and/or the immortalized mammary epithelial cells may be from an endangered species, e.g., an endangered mammal.

In some embodiments, the live primary mammary epithelial cells, the mixed population of live primary mammary epithelial cells, mammary myoepithelial cells and mammary progenitor cells, and/or the immortalized mammary epithelial cells may be from a human. In some embodiments, the live primary mammary epithelial cells, the mixed population of live primary mammary epithelial cells, mammary myoepithelial cells and mammary progenitor cells, and/or the immortalized mammary epithelial cells may be from a bovid (e.g., a cow).

In some embodiments, milk produced by the primary mammary epithelial cells (e.g., primary mammary epithelial cells from the isolated live primary mammary epithelial cells and/or the primary mammary epithelial cells from the mixed population of live primary mammary epithelial cells, mammary myoepithelial cells and/or mammary progenitor cells) or the immortalized mammary epithelial cells may be excreted through the apical surface of the cells into the apical compartment.

In some embodiments, a basal compartment may comprise a basal culture medium and the basal culture medium may be in contact with the basal surface of the live primary mammary epithelial cells, the mixed population of live primary mammary epithelial cells, mammary myoepithelial cells and mammary progenitor cells, and/or the immortalized mammary epithelial cells.

In some embodiments, the basal culture medium of the present invention may comprise a carbon source, a chemical buffering system, one or more essential amino acids, one or more vitamins and/or cofactors, and one or more inorganic salts.

In some embodiments, the basal culture medium may comprise a carbon source in an amount from about 1 g/L to about 15 g/L of basal culture medium (e.g., about 1, 2, 3, 4, 5, 6, 7, 8, 9, 10, 11, 12, 13, 14 or 15 g/L or any value or range therein), or about 1, 2, 3, 4, 5 or 6 g/L to about 7, 8, 9, or 10, 11, 12, 13, 14 or 15 g/L of the basal culture medium. Non-limiting examples of a carbon source include glucose and/or pyruvate. For example, in some embodiments, the basal culture medium may comprise glucose in an amount from about 1 g/L to about 12 g/L of basal culture medium, e.g., about 1, 2, 3, 4, 5, 6, 7, 8, 9, 10, 11, or 12 g/L or any value or range therein. In some embodiments, the basal culture medium may comprise glucose in an amount from about 1 g/L to about 6 g/L, about 4 g/L to about 12 g/L, about 2.5 g/L to about 10.5 g/L, about 1.5 g/L to about 11.5 g/L, or about 2 g/L to about 10 g/L of basal culture medium. In some embodiments, the basal culture medium may comprise glucose in an amount from about 1, 2, 3, or 4 g/L to about 5, 6, 7, 8, 9, 10, 11, or 12 g/L or about 1, 2, 3, 4, 5, or 6 g/L to about 7, 8, 9, 10, 11, or 12 g/L. In some embodiments, the basal culture medium may comprise pyruvate in an amount from about 5 g/L to about 15 g/L of basal culture medium, e.g., about 5, 6, 7, 8, 9, 10, 11, 12, 13, 14, or 15 g/L or any value or range therein. In some embodiments, the basal culture medium may comprise pyruvate in an amount from about 5 g/L to about 14.5 g/L, about 10 g/L to about 15 g/L, about 7.5 g/L to about 10.5 g/L, about 5.5 g/L to about 14.5 g/L, or about 8 g/L to about 10 g/L of basal culture medium. In some embodiments, the basal culture medium may comprise pyruvate in an amount from about 5, 6, 7, or 8 g/L to about 9, 10, 11, 12, 13, 14 or 15 g/L or about 5, 6, 7, 8, 9, or 10 g/L to about 11, 12, 13, 14 or 15 g/L.

In some embodiments, the basal culture medium may comprise a chemical buffering system in an amount from about 1 g/L to about 4 g/L (e.g., about 1, 1.5, 2, 2.5, 3, 3.5, or 4 g/L or any value or range therein) of basal culture medium or about 10 mM to about 25 mM (e.g., about 10, 11, 12, 13, 14, 15, 16, 17, 18, 19, 20, 21, 22, 23, 24, or 25 mM or any value or range therein). In some embodiments, the chemical buffering system may include, but is not limited to, sodium bicarbonate and/or 4-(2-hydroxyethyl)-1-piperazineethanesulfonic acid (HEPES). For example, in some embodiments, the basal culture medium may comprise sodium bicarbonate in an amount from about 1 g/L to about 4 g/L of basal culture medium, e.g., about 1, 1.5, 2, 2.5, 3, 3.5, or 4 g/L or any value or range therein. In some embodiments, the basal culture medium may comprise sodium bicarbonate in an amount from about 1 g/L to about 3.75 g/L, about 1.25 g/L to about 4 g/L, about 2.5 g/L to about 3 g/L, about 1.5 g/L to about 4 g/L, or about 2 g/L to about 3.5 g/L of basal culture medium. In some embodiments, the basal culture medium may comprise HEPES in an amount from about 10 mM to about 25 mM, e.g., about 10, 11, 12, 13, 14, 15, 16, 17, 18, 19, 20, 21, 22, 23, 24, or 25 mM or any value or range therein. In some embodiments, the basal culture medium may comprise HEPES in an amount from about 11 mM to about 25 mM, about 10 mM to about 20 mM, about 12.5 mM to about 22.5 mM, about 15 mM to about 20.75 mM, or about 10 mM to about 20 mM.

In some embodiments, the basal culture medium may comprise one or more essential amino acids in an amount from about 0.5 mM to about 5 mM (e.g., about 0.5, 1, 1.5, 2, 2.5, 3, 3.5, 4, 4.5, or 5 mM or any value or range therein) or about 0.5, 1, 1.5, 2 mM to about 2.5, 3, 3.5, 4, 4.5, or 5 mM. In some embodiments, the one or more essential amino acids may be, for example, arginine and/or cysteine. For example, in some embodiments, the basal culture medium may comprise arginine in an amount from about 0.5 mM to about 5 mM, e.g., about 0.5, 1, 1.5, 2, 2.5, 3, 3.5, 4, 4.5, or 5 mM or any value or range therein. In some embodiments, the basal culture medium may comprise arginine in an amount from about 0.5 mM to about 4.75 mM, about 2 mM to about 3.5 mM, about 0.5 mM to about 3.5 mM, about 1 mM to about 5 mM, or about 3.5 mM to about 5 mM. For example, in some embodiments, the basal culture medium may comprise cysteine in an amount from about 0.5 mM to about 5 mM, e.g., about 0.5, 1, 1.5, 2, 2.5, 3, 3.5, 4, 4.5, or 5 mM or any value or range therein. In some embodiments, the basal culture medium may comprise cysteine in an amount from about 0.5 mM to about 4.75 mM, about 2 mM to about 3.5 mM, about 0.5 mM to about 3.5 mM, about 1 mM to about 5 mM, or about 3.5 mM to about 5 mM.

In some embodiments, the basal culture medium may comprise one or more vitamins and/or cofactors in an amount from about 0.01 μM to about 50 μM (e.g., about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 3, 4, 5, 6, 7, 8, 9, 10, 12.5, 15, 17.5, 20, 25, 30, 35, 40, 45, 46, 47, 48, 49, 49.025, 49.05, 49.075, or 50 μM or any value or range therein) or about 0.01, 0.02, 0.03, 0.04, 0.05, 0.06, 0.07, 0.08, 0.09, 0.1, 0.2, 0.3, 0.4, 0.5, 0.6, 0.7, 0.8, or 0.9 μM to about 1, 1.1, 1.2, 1.3, 1.4, 1.5, 1.6, 1.7, 1.8, 1.9, 2, 2.1, 2.2, 2.3, 2.4, 2.5, 3, 4, 5, 6 μM or about 0.02, 0.025, 0.05, 0.075, 1, 1.5, 2, 3, 4, 5, 6, 7, 8, 9, 10 μM to about 12.5, 15, 17.5, 20, 25, 30, 35, 40, 45, 46, 47, 48, 49, 49.025, 49.05, 49.075, or 50 μM. In some embodiments, one or more vitamins and/or cofactors may include, but are not limited to, thiamine and/or riboflavin. For example, in some embodiments, the basal culture medium may comprise thiamine in an amount from about 0.025 μM to about 50 μM, e.g., about 0.025, 0.05, 0.075, 1, 1.5, 2, 3, 4, 5, 6, 7, 8, 9, 10, 12.5, 15, 17.5, 20, 25, 30, 35, 40, 45, 46, 47, 48, 49, 49.025, 49.05, 49.075, or 50 μM or any value or range therein. In some embodiments, the basal culture medium may comprise thiamine in an amount from about 0.025 μM to about 45.075 μM, about 1 μM to about 40 μM, about 5 μM to about 35.075 μM, about 10 μM to about 50 μM, or about 0.05 μM to about 45.5 μM. In some embodiments, the basal culture medium may comprise riboflavin in an amount from about 0.01 μM to about 3 μM, e.g., about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, or 3 μM or any value or range therein. In some embodiments, the basal culture medium may comprise riboflavin in an amount from about 0.01 μM to about 2.05 μM, about 1 μM to about 2.95 μM, about 0.05 μM to about 3 μM, about 0.08 μM to about 1.55 μM, or about 0.05 μM to about 2.9 μM.

In some embodiments, the basal culture medium may comprise one or more inorganic salts in an amount from about 100 mg/L to about 150 mg/L of basal culture medium (e.g., about 100, 105, 110, 115, 120, 125, 130, 135, 140, 145, or 150 mg/L or any value or range therein) or about 100 mg/L to about 150 mg/L of basal culture medium (e.g., about 100, 105, 110, 115, 120, 125, 130, 135, 140, 145, or 150 mg/L or any value or range therein). In some embodiments, one or more inorganic salts may include, but are not limited to, calcium and/or magnesium. For example, in some embodiments, the basal culture medium may comprise calcium in an amount from about 100 mg/L to about 150 mg/L of basal culture medium, e.g., about 100, 105, 110, 115, 120, 125, 130, 135, 140, 145, or 150 mg/L or any value or range therein. In some embodiments, the basal culture medium may comprise arginine in an amount from about 100 mg/L to about 125 mg/L, about 105 mg/L to about 150 mg/L, about 120 mg/L to about 130 mg/L, or about 100 mg/L to about 145 mg/L of basal culture medium. In some embodiments, the basal culture medium may comprise magnesium in an amount from about 0.01 mM to about 1 mM, e.g., about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 0.91, 0.92, 0.93, 0.94, 0.95, 0.96, 0.97, 0.98, 0.99, or 1 mM or any value or range therein. In some embodiments, the basal culture medium may comprise magnesium in an amount from about 0.05 mM to about 1 mM, about 0.01 mM to about 0.78 mM, about 0.5 mM to about 1 mM, about 0.03 mM to about 0.75 mM, or about 0.25 mM to about 0.95 mM.

In some embodiments, the carbon source, chemical buffering system, one or more essential amino acids, one or more vitamins and/or cofactors, and/or one or more inorganic salts may be food grade. In some embodiments, the basal culture medium may be lactogenic culture medium, e.g., the basal culture medium may further comprise prolactin (e.g., mammalian prolactin, e.g., human prolactin). For example, in some embodiments, the basal culture medium may comprise prolactin (or prolactin may be added) in an amount from about 20 ng/mL to about 200 ng/L of basal culture medium, e.g., about 20, 30, 40, 50, 60, 70, 80, 90, 100, 110, 120, 130, 140, 150, 160, 170, 180, 190, or 200 ng/mL or any value or range therein. In some embodiments, the basal culture medium may comprise prolactin (or prolactin may be added) in an amount from about 20 ng/mL to about 195 ng/mL, about 50 ng/mL to about 150 ng/mL, about 25 ng/mL to about 175 ng/mL, about 45 ng/mL to about 200 ng/mL, or about 75 ng/mL to about 190 ng/mL of basal culture medium. In some embodiments, the basal culture medium may further comprise other factors to improve efficiency, including, but not limited to, insulin, an epidermal growth factor, and/or a hydrocortisone.

In some embodiments, the scaffold of the present invention may be fabricated as a 2-dimensional surface, a 3-dimensional micropatterned surface, and/or as a cylindrical structure that can be assembled into bundles. Non-limiting examples of a 2-dimensional surface scaffold include a transwell filter. Non-limiting examples of a 3-dimensional micropatterned surface include a microstructured bioreactor, a decellularized tissue (e.g, a decellularized mammary gland) and/or a cylindrical structure that can be assembled into bundles (e.g., a hollow fiber bioreactor). In some embodiments, the scaffold of the present invention may be porous.

In some embodiments, the top surface of the scaffold may be coated with one or more extracellular matrix proteins. Non-limiting examples of extracellular matrix proteins include collagen, laminin, entactin, tenascin, and/or fibronectin. In some embodiments, the scaffold may comprise a natural polymer, a biocompatible synthetic polymer, a synthetic peptide, and/or a composite derived from any combination thereof. In some embodiments, a natural polymer useful with this invention may include, but is not limited to, collagen, chitosan, cellulose, agarose, alginate, gelatin, elastin, heparan sulfate, chondroitin sulfate, keratan sulfate, and/or hyaluronic acid. In some embodiments, a biocompatible synthetic polymer useful with this invention may include, but is not limited to, polysulfone, polyvinylidene fluoride, polyethylene co-vinyl acetate, polyvinyl alcohol, sodium polyacrylate, an acrylate polymer, and/or polyethylene glycol.

### Methods

The present invention further provides methods of making a live cell construct, methods of producing milk in culture, and/or methods of producing a modified primary mammary epithelial cell or an immortalized mammary epithelial cell, e.g., for use in the present invention.

Thus, in some embodiments, the present invention provides a method of producing milk in culture, the method comprising culturing the live cell construct of the present invention, thereby producing milk in culture.

In some embodiments, the present invention provides a method of making a live cell construct for producing milk in culture, the method comprising (a) isolating primary mammary epithelial cells, myoepithelial cells and mammary progenitor cells from mammary explants from mammary tissue (e.g., breast, udder, teat tissue), to produce isolated mammary epithelial cells, myoepithelial cells and mammary progenitor cells; (b) culturing the isolated primary mammary epithelial cells, myoepithelial cells and mammary progenitor cells to produce a mixed population of primary mammary epithelial cells, mammary myoepithelial cells and mammary progenitor cells; (c) cultivating the mixed population of (b) on a scaffold, the scaffold having an upper surface and lower surface, to produce a polarized, continuous (i.e., confluent) monolayer of primary mammary epithelial cells, myoepithelial cells and mammary progenitor cells of the mixed population on the upper surface of the scaffold, wherein the polarized, continuous monolayer comprises an apical surface and a basal surface, thereby producing a live cell construct for producing milk in culture.

In some embodiments, the present invention provides a method of making a live cell construct for producing milk in culture, the method comprising: a) isolating primary mammary epithelial cells, myoepithelial cells, and/or mammary progenitor cells from mammary explants from mammary tissue (e.g., breast, udder, teat tissue), to produce isolated mammary epithelial cells, myoepithelial cells, and/or mammary progenitor cells; (b) culturing the isolated primary mammary epithelial cells, myoepithelial cells, and/or mammary progenitor cells to produce a mixed population of primary mammary epithelial cells, mammary myoepithelial cells and mammary progenitor cells; (c) sorting the mixed population of primary mammary epithelial cells, myoepithelial cells, and/or mammary progenitor cells (e.g., selecting the primary mammary epithelial cells) to produce a population of primary mammary epithelial cells; and (d) cultivating the population of primary mammary epithelial on a scaffold, the scaffold having an upper surface and lower surface, to produce a polarized, continuous (i.e., confluent) monolayer of primary mammary epithelial cells on the upper surface of the scaffold, wherein the polarized, continuous monolayer comprises an apical surface and a basal surface, thereby producing a live cell construct for producing milk in culture.

In some embodiments, the present invention provides a method of making a live cell construct for producing milk in culture, the method comprising (a) culturing immortalized mammary epithelial cells to produce increased numbers of immortalized mammary epithelial cells; (b) cultivating the immortalized mammary epithelial cells of (a) on a scaffold, the scaffold having an upper surface and lower surface, to produce a polarized, continuous (i.e., confluent) monolayer of immortalized mammary epithelial cells on the upper surface of the scaffold, wherein the polarized, continuous monolayer comprises an apical surface and a basal surface, thereby producing a live cell construct for producing milk in culture.

In some embodiments, mammary tissue may be from breast tissue, udder tissue, and/or teat tissue of a mammal. Mammary tissue may be from any mammal, e.g., a primate (e.g., chimpanzee, orangutan, gorilla, monkey (e.g., Old World, New World), lemur, human), a dog, a cat, a rabbit, a mouse, a rat, a horse, a cow, a goat, a sheep, an ox (e.g., Bos spp.), a pig, a deer, a musk deer, a bovid, a whale, a dolphin, a hippopotamus, an elephant, a rhinoceros, a giraffe, a zebra, a lion, a cheetah, a tiger, a panda, a red panda, and an otter. In some embodiments, the mammary tissue may be from an endangered species, e.g., an endangered mammal. In some embodiments, the mammary tissue may be from a human. In some embodiments, the mammary tissue may be from a bovid (e.g., a cow).

In some embodiments, the culturing and/or cultivating is carried out at a temperature of about 35° C. to about 39° C. (e.g., a temperature of about 35° C., 35.5° C., 36° C., 36.5° C., 37° C., 37.5° C., 38° C., 38.5° C. or about 39° C., or any value or range therein, e.g., about 35° C. to about 38° C., about 36° C. to about 39° C., about 36.5° C. to about 39° C., about 36.5° C. to about 37.5° C., or about 36.5° C. to about 38° C.). In some embodiments, methods of the present invention may further comprise wherein the culturing is carried out at a temperature of about 37° C.

In some embodiments, the culturing and/or cultivating is carried out at an atmospheric concentration of CO₂ of about 4% to about 6%, e.g., an atmospheric concentration of CO₂ of about 4%, 4.25%, 4.5%, 4.75%, 5%, 5.25%, 5.5%, 5.75%, or 6% or any value or range therein, e.g., about 4% to about 5.5%, about 4.5% to about 6%, about 4.5% to about 5.5%, or about 5% to about 6%). In some embodiments, methods of the present invention may further comprise wherein the culturing is carried out at an atmospheric concentration of CO₂ of about 5%.

In some embodiments, the culturing and/or cultivating may comprise culturing and/or cultivating in a culture medium that is exchanged about every day to about every 10 days (e.g., every 1 day, every 2 days, every 3 days, every 4 days, every 5 days, every 6 days, every 7 days, every 8 days, every 9 days, every 10 days, or any value or range therein, e.g., about every day to every 3 days, about every 3 days to every 10 days, about every 2 days to every 5 days). In some embodiments, the culturing and/or cultivating may further comprise culturing in a culture medium that is exchanged about every day to about every few hours to about every 10 days, e.g., about every 1, 2, 3, 4, 5, 6, 7, 8, 9, 10, 11, 12, 13, 14, 15, 16, 17, 18, 19, 20, 21, 22, 23, or 24 hours to about every 1, 2, 3, 4, 5, 6, 7, 8, 9, or 10 days or any value or range therein. For example, in some embodiments, the culturing and/or cultivating may further comprise culturing and/or cultivating in a culture medium that is exchanged about every 12 hours to about every 10 days, about every 10 hours to about every 5 days, or about every 5 hours to about every 3 days.

In some embodiments, the monolayer of the live cell construct made by the methods of the invention for producing milk in culture may be adjacent to the upper surface of the scaffold.

In some embodiments, the live cell construct made by the methods of the invention for producing milk in culture may further comprise an apical compartment that is adjacent to the apical surface of the monolayer.

In some embodiments, the live cell construct made by the methods of the invention for producing milk in culture may comprise a basal compartment that is adjacent to the lower surface of the scaffold.

In some embodiments, a method of making a live cell construct for producing milk in culture of the present invention, prior to culturing immortalized mammary epithelial cells, may further comprise: (i) isolating primary mammary epithelial cells, myoepithelial cells, and/or mammary progenitor cells from mammary explants from mammary tissue (e.g., breast, udder, and/or teat tissue), to produce isolated mammary epithelial cells, myoepithelial cells, and/or mammary progenitor cells; (ii) culturing the isolated primary mammary epithelial cells, myoepithelial cells, and/or mammary progenitor cells to produce a mixed population of primary mammary epithelial cells, mammary myoepithelial cells and mammary progenitor cells; (iii) sorting the mixed population of primary mammary epithelial cells, myoepithelial cells, and/or mammary progenitor cells (e.g., selecting the primary mammary epithelial cells) to produce a population of primary mammary epithelial cells; and (iv) stably introducing (e.g., transfecting/transducing) one or more cells of the population of primary mammary epithelial cells of (iii) with (1) one or more nucleic acids encoding human telomerase reverse transcriptase (hTERT) or simian virus 40 (SV40), or with (2) a small hairpin RNA (shRNA) to p16 (Inhibitor of Cyclin-Dependent Kinase 4) (p16(INK4)) and Master Regulator of Cell Cycle Entry and Proliferative Metabolism (c-MYC) to produce immortalized mammary epithelial cells. In some embodiments, the immortalized cell line may be stably introduced (e.g., transfected/transduced) with (1) one or more nucleic acids encoding hTERT or SV40, and/or (2) a small hairpin RNA (shRNA) to p16 (Inhibitor of Cyclin-Dependent Kinase 4) (p16(INK4)) and Master Regulator of Cell Cycle Entry and Proliferative Metabolism (c-MYC).

In some embodiments, a method of making a live cell construct for producing milk in culture may further comprise storing cells or populations of cells of the present invention (e.g., the live primary mammary epithelial cells, the mixed population primary mammary epithelial cells, myoepithelial cells and mammary progenitor cells, and/or the immortalized mammary epithelial cells) prior to cultivating on a scaffold, optionally wherein the storing is in a freezer or in liquid nitrogen. Storage temperature may depend on the desired storage length. For example, freezer temperature (e.g., storage at a temperature of about 0° C. to about −80° C. or less, e.g., about 0° C., −10° C., −20° C., −30° C., −40° C., −50° C., −60° C., −70° C., −80° C., −90° C., −100° C. or any value or range therein) may be used if the cells are to be used within 6 months (e.g., within 1, 2, 3, 4, 5, or 6 months). For example, liquid nitrogen may be used (e.g., storage at a temperature of −100° C. or less (e.g., about −100° C., −110° C., −120° C., −130, −140, −150, −160, −170, −180, −190° C., −200° C., or less) for longer term storage (e.g., storage of 6 months or longer, e.g., 6, 7, 8, 9, 10, 11, or 12 months, or 1, 2, 3, 4, 5, 6 or more years).

In some embodiments, a method of making a live cell construct for producing milk in culture may comprise wherein the isolating and sorting is via fluorescence-activated cell sorting, magnetic-activated cell sorting, and/or microfluidic cell sorting.

In some embodiments, the present invention provides a method of producing milk in culture comprising, culturing a live cell construct comprising (a) a scaffold comprising an upper surface and a lower surface and a continuous (i.e., confluent) polarized monolayer of live primary mammary epithelial cells, a continuous polarized monolayer of a mixed population of live primary mammary epithelial cells, mammary myoepithelial cells and mammary progenitor cells, and/or a continuous polarized monolayer of live immortalized mammary epithelial cells having an apical surface and a basal surface, wherein the continuous polarized monolayer of live primary mammary epithelial cells, the continuous polarized monolayer of the mixed population of live primary mammary epithelial cells, mammary myoepithelial cells and mammary progenitor cells and/or the continuous polarized monolayer of live immortalized mammary epithelial cells are located on the upper surface of scaffold, (b) a basal compartment and an apical compartment, wherein the lower surface of the scaffold is adjacent to the basal compartment and the apical surface of the continuous polarized monolayer of live primary mammary epithelial cells, the continuous polarized monolayer of the mixed population of live primary mammary epithelial cells, mammary myoepithelial cells and mammary progenitor cells, and/or the continuous polarized monolayer of live immortalized mammary epithelial cells is adjacent to the apical compartment, wherein the continuous polarized monolayer of live primary epithelial mammary cells, the live primary epithelial mammary cells of the continuous polarized monolayer of the mixed population of live primary mammary epithelial cells, mammary myoepithelial cells and mammary progenitor cells, or the continuous polarized monolayer of immortalized mammary epithelial cells excretes milk through its apical surface into the apical compartment, thereby producing milk in culture.

In some embodiments, the monolayer of the live cell construct for the methods of producing milk in culture may be adjacent to the upper surface of the scaffold.

In some embodiments, the live cell construct for the methods of producing milk in culture may further comprise an apical compartment that is adjacent to the apical surface of the monolayer.

In some embodiments, the live cell construct for the methods of producing milk in culture may comprise a basal compartment that is adjacent to the lower surface of the scaffold.

In some embodiments, a method of producing milk in culture of the present invention may further comprise a basal compartment comprising a basal culture medium and the basal culture medium may be in contact with the basal surface of the continuous polarized monolayer of primary mammary epithelial cells, with the basal surface of the continuous polarized the monolayer of the mixed population, or with the basal surface of the continuous polarized monolayer of live immortalized mammary epithelial cells. The basal culture medium may comprise a carbon source, a chemical buffering system, one or more essential amino acids, one or more vitamins and/or cofactors, and one or more inorganic salts.

In some embodiments, the basal culture medium may comprise a carbon source in an amount from about 1 g/L to about 15 g/L of basal culture medium (e.g., about 1, 2, 3, 4, 5, 6, 7, 8, 9, 10, 11, 12, 13, 14 or 15 g/L or any value or range therein), or about 1, 2, 3, 4, 5 or 6 g/L to about 7, 8, 9, or 10, 11, 12, 13, 14 or 15 g/L of the basal culture medium. In some embodiments, the carbon source may include, but is not limited to, be glucose and/or pyruvate. For example, in some embodiments, the basal culture medium may comprise glucose in an amount from about 1 g/L to about 12 g/L of basal culture medium, e.g., about 1, 2, 3, 4, 5, 6, 7, 8, 9, 10, 11, or 12 g/L or any value or range therein. In some embodiments, the basal culture medium may comprise glucose in an amount from about 1 g/L to about 6 g/L, about 4 g/L to about 12 g/L, about 2.5 g/L to about 10.5 g/L, about 1.5 g/L to about 11.5 g/L, or about 2 g/L to about 10 g/L of basal culture medium. In some embodiments, the basal culture medium may comprise pyruvate at an amount of about 5 g/L to about 15 g/L of basal culture medium, e.g., about 5, 6, 7, 8, 9, 10, 11, 12, 13, 14, or 15 g/L or any value or range therein. In some embodiments, the basal culture medium may comprise pyruvate in an amount from about 5 g/L to about 14.5 g/L, about 10 g/L to about 15 g/L, about 7.5 g/L to about 10.5 g/L, about 5.5 g/L to about 14.5 g/L, or about 8 g/L to about 10 g/L of basal culture medium.

In some embodiments, the basal culture medium may comprise a chemical buffering system in an amount from about 1 g/L to about 4 g/L (e.g., about 1, 1.5, 2, 2.5, 3, 3.5, or 4 g/L or any value or range therein) of basal culture medium or about 10 mM to about 25 mM (e.g., about 10, 11, 12, 13, 14, 15, 16, 17, 18, 19, 20, 21, 22, 23, 24, or 25 mM or any value or range therein). In some embodiments, the chemical buffering system may include, but is not limited to, sodium bicarbonate and/or HEPES. For example, in some embodiments, the basal culture medium may comprise sodium bicarbonate in an amount from about 1 g/L to about 4 g/L of basal culture medium, e.g., about 1, 1.5, 2, 2.5, 3, 3.5, or 4 g/L or any value or range therein. In some embodiments, the basal culture medium may comprise sodium bicarbonate in an amount from about 1 g/L to about 3.75 g/L, about 1.25 g/L to about 4 g/L, about 2.5 g/L to about 3 g/L, about 1.5 g/L to about 4 g/L, or about 2 g/L to about 3.5 g/L of basal culture medium. In some embodiments, the basal culture medium may comprise HEPES in an amount from about 10 mM to about 25 mM, e.g., about 10, 11, 12, 13, 14, 15, 16, 17, 18, 19, 20, 21, 22, 23, 24, or 25 mM or any value or range therein. In some embodiments, the basal culture medium may comprise HEPES in an amount from about 11 mM to about 25 mM, about 10 mM to about 20 mM, about 12.5 mM to about 22.5 mM, about 15 mM to about 20.75 mM, or about 10 mM to about 20 mM.

In some embodiments, the basal culture medium may comprise one or more essential amino acids in an amount from about 0.5 mM to about 5 mM (e.g., about 0.5, 1, 1.5, 2, 2.5, 3, 3.5, 4, 4.5, or 5 mM or any value or range therein) or about 0.5, 1, 1.5, 2 mM to about 2.5, 3, 3.5, 4, 4.5, or 5 mM. In some embodiments, exemplary one or more essential amino acids may be arginine and/or cysteine. For example, in some embodiments, the basal culture medium may comprise arginine in an amount from about 0.5 mM to about 5 mM, e.g., about 0.5, 1, 1.5, 2, 2.5, 3, 3.5, 4, 4.5, or 5 mM or any value or range therein. In some embodiments, the basal culture medium may comprise arginine in an amount from about 0.5 mM to about 4.75 mM, about 2 mM to about 3.5 mM, about 0.5 mM to about 3.5 mM, about 1 mM to about 5 mM, or about 3.5 mM to about 5 mM. For example, in some embodiments, the basal culture medium may comprise cysteine in an amount from about 0.5 mM to about 5 mM, e.g., about 0.5, 1, 1.5, 2, 2.5, 3, 3.5, 4, 4.5, or 5 mM or any value or range therein. In some embodiments, the basal culture medium may comprise cysteine in an amount from about 0.5 mM to about 4.75 mM, about 2 mM to about 3.5 mM, about 0.5 mM to about 3.5 mM, about 1 mM to about 5 mM, or about 3.5 mM to about 5 mM.

In some embodiments, the basal culture medium may comprise one or more vitamins and/or cofactors in an amount from about 0.01 μM to about 50 μM (e.g., about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 3, 4, 5, 6, 7, 8, 9, 10, 12.5, 15, 17.5, 20, 25, 30, 35, 40, 45, 46, 47, 48, 49, 49.025, 49.05, 49.075, or 50 μM or any value or range therein) or about 0.01, 0.02, 0.03, 0.04, 0.05, 0.06, 0.07, 0.08, 0.09, 0.1, 0.2, 0.3, 0.4, 0.5, 0.6, 0.7, 0.8, or 0.9 μM to about 1, 1.1, 1.2, 1.3, 1.4, 1.5, 1.6, 1.7, 1.8, 1.9, 2, 2.1, 2.2, 2.3, 2.4, 2.5, 3, 4, 5, 6 μM or about 0.02, 0.025, 0.05, 0.075, 1, 1.5, 2, 3, 4, 5, 6, 7, 8, 9, 10 μM to about 12.5, 15, 17.5, 20, 25, 30, 35, 40, 45, 46, 47, 48, 49, 49.025, 49.05, 49.075, or 50 μM. In some embodiments, one or more vitamins and/or cofactors may include, but is not limited to, thiamine and/or riboflavin. For example, in some embodiments, the basal culture medium may comprise thiamine in an amount from about 0.025 μM to about 50 μM, e.g., 0.025, 0.05, 0.075, 1, 1.5, 2, 3, 4, 5, 6, 7, 8, 9, 10, 12.5, 15, 17.5, 20, 25, 30, 35, 40, 45, 46, 47, 48, 49, 49.025, 49.05, 49.075, or 50 μM or any value or range therein. In some embodiments, the basal culture medium may comprise thiamine in an amount from about 0.025 μM to about 45.075 μM, about 1 μM to about 40 μM, about 5 μM to about 35.075 μM, about 10 μM to about 50 μM, or about 0.05 μM to about 45.5 μM. In some embodiments, the basal culture medium may comprise riboflavin in an amount from about 0.01 μM to about 3 μM, e.g., 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, or 3 μM or any value or range therein. In some embodiments, the basal culture medium may comprise riboflavin in an amount from about 0.01 μM to about 2.05 μM, about 1 μM to about 2.95 μM, about 0.05 μM to about 3 μM, about 0.08 μM to about 1.55 μM, or about 0.05 μM to about 2.9 μM.

In some embodiments, the basal culture medium may comprise one or more inorganic salts in an amount from about 100 mg/L to about 150 mg/L of basal culture medium (e.g., about 100, 105, 110, 115, 120, 125, 130, 135, 140, 145, or 150 mg/L or any value or range therein) or about 100 mg/L to about 150 mg/L of basal culture medium (e.g., about 100, 105, 110, 115, 120, 125, 130, 135, 140, 145, or 150 mg/L or any value or range therein). In some embodiments, exemplary one or more inorganic salts may be calcium and/or magnesium. For example, in some embodiments, the basal culture medium may comprise calcium in an amount from about 100 mg/L to about 150 mg/L of basal culture medium, e.g., about 100, 105, 110, 115, 120, 125, 130, 135, 140, 145, or 150 mg/L or any value or range therein. In some embodiments, the basal culture medium may comprise arginine in an amount from about 100 mg/L to about 125 mg/L, about 105 mg/L to about 150 mg/L, about 120 mg/L to about 130 mg/L, or about 100 mg/L to about 145 mg/L of basal culture medium. In some embodiments, the basal culture medium may comprise magnesium in an amount from f about 0.01 mM to about 1 mM, e.g., about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 0.91, 0.92, 0.93, 0.94, 0.95, 0.96, 0.97, 0.98, 0.99, or 1 mM or any value or range therein. In some embodiments, the basal culture medium may comprise magnesium in an amount from about 0.05 mM to about 1 mM, about 0.01 mM to about 0.78 mM, about 0.5 mM to about 1 mM, about 0.03 mM to about 0.75 mM, or about 0.25 mM to about 0.95 mM.

In some embodiments, the carbon source, chemical buffering system, one or more essential amino acids, one or more vitamins and/or cofactors, and/or one or more inorganic salts may be food grade.

In some embodiments, the basal culture medium may be lactogenic culture medium, e.g., the basal culture medium may further comprise prolactin (e.g., mammalian prolactin, e.g., human prolactin). For example, in some embodiments, the basal culture medium may comprise prolactin (or prolactin may be added) in an amount from about 20 ng/mL to about 200 ng/L of basal culture medium, e.g., about 20, 30, 40, 50, 60, 70, 80, 90, 100, 110, 120, 130, 140, 150, 160, 170, 180, 190, or 200 ng/mL or any value or range therein. In some embodiments, the basal culture medium may comprise prolactin (or prolactin may be added) in an amount from about 20 ng/mL to about 195 ng/mL, about 50 ng/mL to about 150 ng/mL, about 25 ng/mL to about 175 ng/mL, about 45 ng/mL to about 200 ng/mL, or about 75 ng/mL to about 190 ng/mL of basal culture medium. In some embodiments, the methods of the present invention may further comprise adding prolactin to the basal culture medium, thereby providing a lactogenic culture medium. In some embodiments, the prolactin may be produced by a microbial cell and/or a human cell expressing a recombinant prolactin (e.g., a prolactin comprising a substitution of a serine residue at position 179 of the prolactin gene with aspartate (S179D), e.g., S179D-prolactin). In some embodiments, adding prolactin to the basal culture medium may comprise conditioning basal culture medium by culturing cells that express and secrete prolactin, and applying the conditioned basal culture medium comprising prolactin to the basal surface of the monolayer of primary mammary epithelial cells, the basal surface of the monolayer of the mixed population, or the basal surface of the monolayer of live immortalized mammary epithelial cells.

In some embodiments, the basal culture medium may further comprise other factors to improve efficiency, including, but not limited to, insulin, an epidermal growth factor, and/or a hydrocortisone. In some embodiments, the methods of the present invention may further comprise adding other factors (e.g., insulin, an epidermal growth factor, and/or a hydrocortisone) to the basal culture medium, e.g., to improve efficiency.

In some embodiments, the methods of the present invention may comprise monitoring the glucose concentration and/or rate of glucose consumption in the basal culture medium and/or in the lactogenic culture medium. In some embodiments, the prolactin may be added when the rate of glucose consumption in the basal culture medium is steady state.

In some embodiments, a method of producing milk in culture may comprise culturing at a temperature of about 35° C. to about 39° C. (e.g., a temperature of about 35° C., 35.5° C., 36° C., 36.5° C., 37° C., 37.5° C., 38° C., 38.5° C. or about 39° C., or any value or range therein, e.g., about 35° C. to about 38° C., about 36% to about 39° C., about 36.5° C. to about 39° C., about 36.5° C. to about 38° C., or about 36.5° C. to about 37.5° C.). In some embodiments, the culturing may be carried out at a temperature of about 37° C.

In some embodiments, a method of producing milk in culture may comprise culturing at an atmospheric concentration of CO₂ of about 4% to about 6%, e.g., an atmospheric concentration of CO₂ of about 4%, 4.25%, 4.5%, 4.75%, 5%, 5.25%, 5.5%, 5.75%, or 6% or any value or range therein, e.g., about 4% to about 5.5%, about 4.5% to about 6%, about 4.5% to about 5.5%, or about 5% to about 6%). In some embodiments, the culturing may be carried out at an atmospheric concentration of CO₂ of about 5%.

In some embodiments, a method of producing milk in culture may comprise monitoring the concentration of dissolved O₂ and CO₂. In some embodiments, the concentration of dissolved O₂ may be maintained between about 10% to about 25% or any value or range therein (e.g., about 10, 11, 12, 13, 14, 15, 16, 17, 18, 19, 20, 21, 22, 23, 24, or 25%). For example, in some embodiments, the concentration of dissolved O₂ may be maintained between about 12% to about 25%, about 15% to about 22%, about 10% to about 20%, about 15%, about 20%, or about 22%. In some embodiments, the concentration of CO₂ may be maintained between about 4% to about 6%, e.g., a concentration of CO₂ of about 4%, 4.25%, 4.5%, 4.75%, 5%, 5.25%, 5.5%, 5.75%, or 6% or any value or range therein, e.g., about 4% to about 5.5%, about 4.5% to about 6%, about 4.5% to about 5.5%, or about 5% to about 6%). In some embodiments, the concentration of CO₂ may be maintained at about 5%.

In some embodiments, a method of producing milk in culture may further comprise applying a transepithelial electrical resistance (TEER) to measure the maintenance of the monolayer of epithelial cells. TEER measures a voltage difference between the fluids (e.g., media) in two compartments (e.g., between the apical and basal compartments), wherein if the barrier between the compartments loses integrity, the fluids in the two compartments may mix. When there is fluid mixing, there will be no voltage difference; a voltage difference indicates that the barrier is intact. Upon detection of a loss of voltage by TEER, a scaffold (e.g., a transwell filter, a microstructured bioreactor, a decellularized tissue, a hollow fiber bioreactor, etc.) may be reinoculated with additional cells and allowed time to reestablish a barrier (e.g., a confluent, continuous monolayer) before resuming methods of the present invention (e.g., milk production).

In some embodiments, a method of producing milk in culture may further comprise storing cells or populations of cells of the present invention (e.g., the live primary mammary epithelial cells, the mixed population primary mammary epithelial cells, myoepithelial cells and mammary progenitor cells, and/or the immortalized mammary epithelial cells) prior to cultivating on a scaffold, optionally wherein the storing is in a freezer or in liquid nitrogen. Storage temperature may depend on the desired storage length. For example, freezer temperature (e.g., storage at a temperature of about 0° C. to about −80° C. or less, e.g., about 0° C., −10° C., −20° C., −30° C., −40° C., −50° C., −60° C., −70° C., −80° C., −90° C., −100° C. or any value or range therein) may be used if the cells are to be used within 6 months (e.g., within 1, 2, 3, 4, 5, or 6 months). For example, liquid nitrogen may be used (e.g., storage at a temperature of −100° C. or less (e.g., about −100° C., −110° C., −120° C., −130, −140, −150, −160, −170, −180, −190° C., −200° C., or less) for longer term storage (e.g., storage of 6 months or longer, e.g., 6, 7, 8, 9, 10, 11, or 12 months, or 1, 2, 3, 4, 5, 6 or more years).

In some embodiments, a method of producing milk in culture may further comprise comprising collecting the milk from the apical compartment to produce collected milk. In some embodiments, the collecting may be via a port, via gravity, and/or via a vacuum. In some embodiments, a vacuum may be attached to a port.

In some embodiments, a method of producing milk in culture may further comprise freezing the collected milk to produce frozen milk and/or lyophilizing the collected milk to produce lyophilized milk.

In some embodiments, a method of producing milk in culture may further comprise packaging the collected milk, the frozen milk and/or the lyophilized milk into a container.

In some embodiments, a method of producing milk in culture may further comprise extracting one or more components from the collected milk. Non-limiting examples of components from the collected milk include milk protein, lipid, carbohydrate, vitamin, and/or mineral contents. In some embodiments, the components from the collected milk may be lyophilized and/or concentrated to produce a lyophilized or a concentrated milk component product. In some embodiments, the components from the collected milk may concentrated by, e.g., membrane filtration and/or reverse osmosis. In some embodiments, the lyophilized or concentrated milk component product may be packaged in a container, optionally wherein the container is sterile and/or a food grade container. In some embodiments, the container may be vacuum-sealed. In some embodiments, the container may be a canister, a jar, a bottle, a bag, a box, or a pouch.

The present invention also provides a method of producing a modified primary mammary epithelial cell or a immortalized mammary epithelial cell, wherein the method comprises introducing into the cell: (a) a polynucleotide encoding a prolactin receptor comprising a modified intracellular signaling domain, optionally wherein the prolactin receptor comprises a truncation wherein position 154 of exon 10 has been spliced to the 3′ sequence of exon 11; (b) a polynucleotide encoding a chimeric prolactin receptor that binds to a ligand, which is capable of activating milk synthesis in the absence of prolactin; (c) a polynucleotide encoding a constitutively or conditionally active prolactin receptor protein, optionally wherein the polynucleotide encodes a constitutively active human prolactin receptor protein comprising a deletion of amino acids 9 through 187 (e.g., a deletion of amino acids 9 through 187, wherein the numbering is based on the reference amino acid sequence of a human prolactin receptor identified as SEQ ID NO:1); (d) a polynucleotide encoding a modified (e.g., recombinant) effector of a prolactin protein comprising (i) a janus kinase-2 (JAK2) tyrosine kinase domain, optionally wherein the JAK2 tyrosine kinase domain may be fused to a signal transducer and activator of transcription-5 (STATS) tyrosine kinase domain (e.g., a polynucleotide encoding a JAK2 tyrosine kinase domain linked to the 3′ end of a polynucleotide encoding the STATS tyrosine kinase domain); and/or (ii) a prolactin receptor intracellular domain fused to a JAK2 tyrosine kinase domain; (e) a loss of function mutation into a circadian related gene PER2 (period circadian protein homolog 2); and/or (f) a polynucleotide encoding one or more glucose transporter genes GLUT1 and/or GLUT12, thereby increasing the rate of nutrient uptake at the basal surface of the monolayer.

In some embodiments, a constitutively active human prolactin receptor protein may comprise a deletion of amino acids 9 through 187, wherein the numbering is based on the reference amino acid sequence of a human prolactin receptor identified as SEQ ID NO:1.

SEQ ID NO: 1:
Human prolactin receptor (GenBank accession number
AAD32032.1)
MKENVASATVFTLLLFLNTCLLNGQLPPGKPEIFKCRSPNKETFTCWWRP
GTDGGLPTNYSLTYHREGETLMHECPDYITGGPNSCHFGKQYTSMWRTYI
MMVNATNQMGSSFSDELYVDVTYIVQPDPPLELAVEVKQPEDRKPYLWIK
WSPPTLIDLKTGWFTLLYEIRLKPEKAAEWEIHFAGQQTEFKILSLHPGQ
KYLVQVRCKPDHGYWSAWSPATFIQIPSDFTMNDTTVWISVAVLSAVICL
IIVWAVALKGYSMVTCIFPPVPGPKIKGFDAHLLEKGKSEELLSALGCQD
FPPTSDYEDLLVEYLEVDDSEDQHLMSVHSKEHPSQGMKPTYLDPDTDSG
RGSCDSPSLLSEKCEEPQANPSTFYDPEVIEKPENPETTHTWDPQCISME
GKIPYFHAGGSKCSTWPLPQPSQHNPRSSYHNITDVCELAVGPAGAPATL
LNEAGKDALKSSQTIKSREEGKATQQREVESFHSETDQDTPWLLPQEKTP
FGSAKPLDYVEIHKVNKDGALSLLPKQRENSGKPKKPGTPENNKEYAKVS
GVMDNNILVLVPDPHAKNVACFEESAKEAPPSLEQNQAEKALANFTATSS
KCRLQLGGLDYLDPACFTHSFH

In some embodiments, a constitutively active human prolactin receptor protein may comprise a deletion of the following amino acids:

VFTLLLFLNTCLLNGQLPPGKPEIFKCRSPNKETFTCWWRPGTDGGLPTN
YSLTYHREGETLMHECPDYITGGPNSCHFGKQYTSMWRTYIMMVNATNQM
GSSFSDELYVDVTYIVQPDPPLELAVEVKQPEDRKPYLWIKWSPPTLIDL
KTGWFTLLYEIRLKPEKAA (e.g., amino acid positions 9
through 187 of SEQ ID NO: 1).

In some embodiments, a loss of function mutation introduced into a circadian related gene PER2 may comprise an 87-amino acid deletion from position 348 to 434 in PER2, wherein the numbering is based on the reference amino acid sequence of a human PER2 identified as SEQ ID NO:2.

SEQ ID NO: 2:
Human Period circadian protein homolog 2 (GenBank
accession number NM 022817)
MNGYAEFPPSPSNPTKEPVEPQPSQVPLQEDVDMSSGSSGHETNENCSTG
RDSQGSDCDDSGKELGMLVEPPDARQSPDTFSLMMAKSEHINPSTSGCSS
DQSSKVDTHKELIKTLKELKVHLPADKKAKGKASTLATLKYALRSVKQVK
ANEEYYQLLMSSEGHPCGADVPSYTVEEMESVTSEHIVKNADMFAVAVSL
VSGKILYISDQVASIFHCKRDAFSDAKFVEFLAPHDVGVFHSFTSPYKLP
LWSMCSGADSFTQECMEEKSFFCRVSVRKSHENEIRYHPFRMTPYLVKVR
DQQGAESQLCCLLLAERVHSGYEAPRIPPEKRIFTTTHTPNCLFQDVDER
AVPLLGYLPQDLIETPVLVQLHPSDRPLMLAIHKKILQSGGQPFDYSPIR
FRARNGEYITLDTSWSSFINPWSRKISFIIGRHKVRVGPLNEDVFAAHPC
TEEKALHPSIQELTEQIHRLL LQPVPHSGSS GYGSLGSNGSHEHLMSQ
NTSSSDSNGHEDSRRRRAEICKNGKTKNRSHYSHESGEQKKKSVTEMQTN
PPAEKKAVPAMEKDSLGVSFPEELACKNQPTCSYQQISCLDSV IRYLES
CNEAATLKRKCEFP ANVPALRSSDKRKATVSPGPHAGEAEPPSRVNSRT
GVGTHLTSLALPGKAESVASLTSQCSYSSTIVHVGDKKPQPELEMVEDAA
SGPESLDCLAGPALACGLSQEKEPFKKLGLTKEVLAAHTQKEEQSFLQKF
KEIRKLSIFQSHCHYYLQERSKGQPSERTAPGLRNTSGIDSPWKKTGKNR
KLKSKRVKPRDSSESTGSGGPVSARPPLVGLNATAWSPSDTSQSSCPAVP
FPAPVPAAYSLPVFPAPGTVAAPPAPPHASFTVPAVPVDLQHQFAVQPPP
FPAPLAPVMAFMLPSYSFPSGTPNLPQAFFPSQPQFPSHPTLTSEMASAS
QPEFPEGGTGAMGTTOATETAAVGADCKPGTSRDQQPKAPLTRDEPSDTQ
NSDALSTSSGLLNLLLNEDLCSASGSAASESLGSGSLGCDASPSGAGSSD
TSHTSKYFGSIDSSENNHKAKMNTGMEESEHFIKCVLQDPIWLLMADADS
SVMMTYQLPSRNLEAVLKEDREKLKLLQKLQPRFTESQKQELREVHQWMQ
TGGLPAAIDVAECVYCENKEKGNICIPYEEDIPSLGLSEVSDTKEDENGS
PLNHRIEEQT

In some embodiments, a loss of function mutation introduced into a circadian related gene PER2 may comprise a deletion of the following amino acids:

CLFQDVDERAVPLLGYLPQDLIETPVLVQLHPSDRPLMLAIHKKILQSGG
QPFDYSPIRFRARNGEYITLDTSWSSFINPWSRKISFIIGRHKV
(e.g., amino acid positions 348 through 434 of
SEQ ID NO: 2).

In some embodiments, a polynucleotide encoding a prolactin receptor comprising a modified intracellular signaling domain, optionally wherein the prolactin receptor comprises a truncation wherein position 154 of exon 10 has been spliced to the 3′ sequence of exon 11, may encode the following amino acid sequence identified as SEQ ID NO:3.

SEQ ID NO: 3: Human isoform 4 of Prolactin
receptor (GenBank accession number AF416619;
Trott et al. 2003 J. Mol. Endocrinol. 30(l): 1-47)
MKENVASATVFTLLLFLNTCLLNGQLPPGKPEIFKCRSPNKETFTCWWRP
GTDGGLPTNYSLTYHREGETLMHECPDYITGGPNSCHFGKQYTSMWRTYI
MMVNATNQMGSSFSDELYVDVTYIVQPDPPLELAVEVKQPEDRKPYLWIK
WSPPTLIDLKTGWFTLLYEIRLKPEKAAEWEIHFAGQQTEFKILSLHPGQ
KYLVQVRCKPDHGYWSAWSPATFIQIPSDFTMNDTTVWISVAVLSAVICL
IIVWAVALKGYSMVTCIFPPVPGPKIKGFDAHLLEKGKSEELLSALGCQD
FPPTSDYEDLLVEYLEVDDSEDQHLMSVHSKEHPSQGDPLMLGASHYKNL
KSYRPRKISSQGRLAVFTKATLTTVQ

In some embodiments, a polynucleotide encoding a modified (e.g., recombinant) effector of a prolactin protein comprising (i) a janus kinase-2 (JAK2) tyrosine kinase domain, optionally wherein the JAK2 tyrosine kinase domain may be fused to a signal transducer and activator of transcription-5 (STATS) tyrosine kinase domain (e.g., a polynucleotide encoding a JAK2 tyrosine kinase domain linked to the 3′ end of a polynucleotide encoding the STATS tyrosine kinase domain) may encode the following amino acid sequence identified as SEQ ID NO:4. Bolded amino acids correspond to the JAK2 kinase domain of amino acid positions 757 through 1129 of a reference human JAK2 amino acid sequence.

SEQ ID NO: 4. STA5A Human signal transducer and
activator of transcription 5A fused at 3′ end to
amino acids 757-1129 of JAK2 human tyrosine-
protein kinase
MAGWIQAQQL QGDALRQMQV LYGQHFPIEV RHYLAQWIES
QPWDAIDLDN PQDRAQATQL LEGLVQELQK KAEHQVGEDG
FLLKIKLGHY ATQLQKTYDR CPLELVRCIR HILYNEQRLV
REANNCSSPA GILVDAMSQK HLQINQTFEE LRLVTQDTEN
ELKKLQQTQE YFIIQYQESL RIQAQFAQLA QLSPQERLSR
ETALQQKQVS LEAWLQREAQ TLQQYRVELA EKHQKTLQLL
RKQQTIILDD ELIQWKRRQQ LAGNGGPPEG SLDVLQSWCE
KLAEIIWQNR QQIRRAEHLC QQLPIPGPVE EMLAEVNATI
TDIISALVTS TFIIEKQPPQ VLKTQTKFAA TVRLLVGGKL
NVHMNPPQVK ATIISEQQAK SLLKNENTRN ECSGEILNNC
CVMEYHQATG TLSAHFRNMS LKRIKRADRR GAESVTEEKF
TVLFESQFSV GSNELVFQVK TLSLPVVVIV HGSQDHNATA
TVLWDNAFAE PGRVPFAVPD KVLWPQLCEA LNMKFKAEVQ
SNRGLTKENL VFLAQKLFNN SSSHLEDYSG LSVSWSQFNR
ENLPGWNYTF WQWFDGVMEV LKKHHKPHWN DGAILGFVNK
QQAHDLLINK PDGTFLLRFS DSEIGGITIA WKFDSPERNL
WNLKPFTTRD FSIRSLADRL GDLSYLIYVF PDRPKDEVFS
KYYTPVLAKA VDGYVKPQIK QVVPEFVNAS ADAGGSSATY
MDQAPSPAVC PQAPYNMYPQ NPDHVLDQDG EFDLDETMDV
ARHVEELLRR PMDSLDSRLS PPAGLFTSAR GSLSLDSQ
RKLQFYEDRH QLPAPKWAEL ANLINNCMDY EPDFRPSFRA
IIRDLNSLFT PDYELLTEND MLPNMRIGAL GFSGAFEDRD
PTQFEERHLK FLQQLGKGNF GSVEMCRYDP LQDNTGEVVA
VKKLQHSTEE HLRDFEREIE ILKSLQHDNI VKYKGVCYSA
GRRNLKLIME YLPYGSLRDY LQKHKERIDH IKLLQYTSQI
CKGMEYLGTK RYIHRDLATR NILVENENRV KIGDFGLTKV
LPQDKEYYKV KEPGESPIFW YAPESLTESK FSVASDVWSF
GVVLYELFTY IEKSKSPPAE FMRMIGNDKQ GQMIVFHLIE
LLKNNGRLPR PDGCPDEIYM IMTECWNNNV NQRPSFRDLA
LRVDQIRDN

Exemplary embodiments are set forth below:
1. A live cell construct comprising,

a scaffold having a top surface and a bottom surface; and

a continuous monolayer of (a) live primary mammary epithelial cells, (b) a mixed population of live primary mammary epithelial cells, mammary myoepithelial cells and mammary progenitor cells, and/or (c) live immortalized mammary epithelial cells on the top surface of the scaffold, the continuous monolayer of (a) live primary mammary epithelial cells, (b) mixed population of live primary mammary epithelial cells mammary myoepithelial cells and mammary progenitor cells, and/or (c) immortalized mammary epithelial cells having an apical surface and a basal surface (e.g., the cells form a polarized and confluent cell monolayer), wherein the construct comprises an apical compartment above and adjacent to the apical surface of the continuous monolayer of the (a) live primary mammary epithelial cells, the (b) mixed population of live primary mammary epithelial cells, mammary myoepithelial cell and mammary progenitor cells, and/or the (c) immortalized mammary epithelial cells and a basal compartment below and adjacent to the bottom surface of the scaffold.

2. The live cell construct of claim 1, wherein milk produced by the primary mammary epithelial cells or immortalized mammary epithelial cells is excreted through the apical surface of the cells into the apical compartment.
3. The live cell construct of claim 1 or claim 2, wherein the basal compartment comprises a basal culture medium and the basal culture medium is in contact with the basal surface of the live primary mammary epithelial cells, the mixed population of live primary mammary epithelial cells, mammary myoepithelial cells and mammary progenitor cells, and/or the immortalized mammary epithelial cells.
4. The live cell construct of claim 3, wherein the basal culture medium comprises a carbon source, a chemical buffering system, one or more essential amino acids, one or more vitamins and/or cofactors, and one or more inorganic salts.
5. The live cell construct of claim 3 or claim 4, wherein the basal culture medium is a lactogenic culture medium and further comprises prolactin.
6. The live cell construct of any one of claims 1 to 5, wherein the scaffold is fabricated as a 2-dimensional surface (e.g., a transwell filter), a 3-dimensional micropatterned surface (e.g., microstructured bioreactor, decellularized tissue), or as a cylindrical structure that can be assembled into bundles (e.g., hollow fiber bioreactor).
7. The live cell construct of any one of claims 1 to 6, wherein the top surface of the scaffold is coated with one or more extracellular matrix proteins.
8. The live cell construct of claim 6, wherein the one or more extracellular matrix proteins are collagen, laminin, entactin, tenascin, and/or fibronectin.
9. The live cell construct of any one of claims 1 to 8, wherein the scaffold comprises a natural polymer, a biocompatible synthetic polymer, a synthetic peptide, and/or a composite derived from any combination thereof.
10. The live cell construct of claim 9, wherein the natural polymer is collagen, chitosan, cellulose, agarose, alginate, gelatin, elastin, heparan sulfate, chondroitin sulfate, keratan sulfate, and/or hyaluronic acid.
11. The live cell construct of claim 9 or claim 10, wherein the biocompatible synthetic polymer may be polysulfone, polyvinylidene fluoride, polyethylene co-vinyl acetate, polyvinyl alcohol, sodium polyacrylate, an acrylate polymer, and/or polyethylene glycol.
12. The live cell construct of any one of claims 1 to 9, wherein said scaffold is porous.
13. The live cell construct of any one of claims 1 to 13, wherein the live primary mammary epithelial cells, the mixed population of live primary mammary epithelial cells, mammary myoepithelial cells and mammary progenitor cells, and/or the immortalized mammary epithelial cells are from a mammal.
14. The live cell construct of any one of claims 1 to 13, wherein the mammal is a primate (e.g., chimpanzee, orangutan, gorilla, monkey (e.g., Old World, New World), lemur, human), a dog, a cat, a rabbit, a mouse, a rat, a horse, a cow, a goat, a sheep, an ox, a pig, a deer, a musk deer, a bovid, a whale, a dolphin, a hippopotamus, an elephant, a rhinoceros, a giraffe, a zebra, a lion, a cheetah, a tiger, a panda, a red panda, and an otter.
15. The live cell construct of any one of claims 1 to 13, wherein the mammal is from an endangered species.
16. A method of producing milk in culture, the method comprising culturing the live cell construct of any one of claims 1 to 15, thereby producing milk in culture.
17. A method of making a live cell construct for producing milk in culture, the method comprising

(a) isolating primary mammary epithelial cells, myoepithelial cells and/or mammary progenitor cells from mammary explants from mammary tissue, to produce isolated mammary epithelial cells, myoepithelial cells and mammary progenitor cells;

(b) culturing the isolated primary mammary epithelial cells, myoepithelial cells and mammary progenitor cells to produce a mixed population of primary mammary epithelial cells, mammary myoepithelial cells and mammary progenitor cells;

(c) cultivating the mixed population of (b) on a scaffold, the scaffold having an upper surface and lower surface, to produce a polarized, continuous (i.e., confluent) monolayer of primary mammary epithelial cells, myoepithelial cells and mammary progenitor cells of the mixed population on the upper surface of the scaffold, wherein the polarized, continuous monolayer comprises an apical surface and a basal surface, thereby producing a live cell construct for producing milk in culture.

18. The method of claim 17, further comprising storing the isolated primary mammary epithelial cells, myoepithelial cells, and/or mammary progenitor cells of (b) prior to cultivating on a scaffold, optionally wherein the storing is in a freezer or in liquid nitrogen.
19. A method of making a live cell construct for producing milk in culture, the method comprising:

a) isolating primary mammary epithelial cells, myoepithelial cells, and/or mammary progenitor cells from mammary explants from mammary tissue (e.g., breast, udder, teat tissue), to produce isolated mammary epithelial cells, myoepithelial cells, and/or mammary progenitor cells;

(b) culturing the isolated primary mammary epithelial cells, myoepithelial cells, and/or mammary progenitor cells to produce a mixed population of primary mammary epithelial cells, mammary myoepithelial cells and mammary progenitor cells;

(c) sorting the mixed population of primary mammary epithelial cells, myoepithelial cells, and/or mammary progenitor cells to produce a population of primary mammary epithelial cells; and

(d) cultivating the population of primary mammary epithelial on a scaffold, the scaffold having an upper surface and lower surface, to produce a polarized, continuous (i.e., confluent) monolayer of primary mammary epithelial cells on the upper surface of the scaffold, wherein the polarized, continuous monolayer comprises an apical surface and a basal surface, thereby producing a live cell construct for producing milk in culture

20. A method of making a live cell construct for producing milk in culture, the method comprising

(a) culturing immortalized mammary epithelial cells to produce increased numbers of immortalized mammary epithelial cells;

(b) cultivating the immortalized mammary epithelial cells of (a) on a scaffold, the scaffold having an upper surface and lower surface, to produce a polarized, continuous (i.e., confluent) monolayer of immortalized mammary epithelial cells on the upper surface of the scaffold, wherein the polarized, continuous monolayer comprises an apical surface and a basal surface, thereby producing a live cell construct for producing milk in culture.

21. The method of claim 20, wherein prior to culturing immortalized mammary epithelial cells the method comprises:

(i) isolating primary mammary epithelial cells, myoepithelial cells, and/or mammary progenitor cells from mammary explants from mammary tissue (e.g., breast, udder, teat tissue), to produce isolated mammary epithelial cells, myoepithelial cells, and/or mammary progenitor cells;

(ii) culturing the isolated primary mammary epithelial cells, myoepithelial cells, and/or mammary progenitor cells to produce a mixed population of primary mammary epithelial cells, mammary myoepithelial cells and mammary progenitor cells;

(iii) sorting the mixed population of primary mammary epithelial cells, myoepithelial cells, and/or mammary progenitor cells to produce a population of primary mammary epithelial cells; and

(iv) stably transfecting one or more cells of the population of primary mammary epithelial cells of (iii) with one or more nucleic acids encoding hTERT or SV40; or transducing with a small hairpin RNA (shRNA) to p16 Inhibitor of Cyclin-Dependent Kinase 4) (p16(INK4)) and Master Regulator of Cell Cycle Entry and Proliferative Metabolism (c-MYC) to produce immortalized mammary epithelial cells.

22. The method of claim 20 or 21, wherein the immortalized cell line is stably transfected with one or more nucleic acids encoding hTERT or SV40; or transduced with (a) a small hairpin RNA (shRNA) to p16 Inhibitor of Cyclin-Dependent Kinase 4) (p16(INK4)) and (b) Master Regulator of Cell Cycle Entry and Proliferative Metabolism (c-MYC).
23. The method of any one of claims 19 to 22, further comprising storing the population of primary mammary epithelial cells or the immortalized mammary epithelial cells prior to cultivating on a scaffold, optionally wherein the storing is in a freezer or in liquid nitrogen.
24. The method of any one of claims 17 to 23, wherein the basal surface of the monolayer is adjacent to the upper surface of the scaffold.
25. The method of any one of claims 17 to 24, wherein the live cell construct comprises an apical compartment that is adjacent to the apical surface of the monolayer.
26. The method of any one of claims 17 to 25, wherein the live cell construct comprises a basal compartment that is adjacent to the lower surface of the scaffold.
27. The method of any one of claims 17 to 26, wherein the culturing is carried out at a temperature of about 35° C. to about 39° C., optionally about 37° C.
28. The method of any one of claims 17 to 27, wherein the culturing is carried out at an atmospheric concentration of CO2 of about 4% to about 6%, optionally about 5%.
29. The method of any one of claims 17 to 28, wherein the culturing of (b) comprises culturing in a culture medium that is exchanged about every day to about every 10 days, optionally about every day to about every 3 days.
30. The method of any one of claims 19 to 29, wherein the isolating and sorting is via fluorescence-activated cell sorting, magnetic-activated cell sorting, and/or microfluidic cell sorting.
31. A method of producing milk in culture comprising, culturing a live cell construct comprising

(a) a scaffold comprising an upper surface and a lower surface and a continuous (i.e., confluent) polarized monolayer of live primary mammary epithelial cells, a continuous polarized monolayer of a mixed population of live primary mammary epithelial cells, mammary myoepithelial cells and mammary progenitor cells, and/or a continuous polarized monolayer of live immortalized mammary epithelial cells having an apical surface and a basal surface, wherein the continuous polarized monolayer of live primary mammary epithelial cells, the continuous polarized monolayer of the mixed population of live primary mammary epithelial cells, mammary myoepithelial cells and mammary progenitor cells and/or the continuous polarized monolayer of live immortalized mammary epithelial cells are located on the upper surface of scaffold,

(b) a basal compartment and an apical compartment, wherein the lower surface of the scaffold is adjacent to the basal compartment and the apical surface of the monolayer of live primary mammary epithelial cells, the monolayer of the mixed population of live primary mammary epithelial cells, mammary myoepithelial cells and mammary progenitor cells, and/or the monolayer of live immortalized mammary epithelial cells is adjacent to the apical compartment,

wherein the monolayer of live primary epithelial mammary cells, the live primary epithelial mammary cells of the monolayer of the mixed population of live primary mammary epithelial cells, mammary myoepithelial cells and mammary progenitor cells, or the monolayer of immortalized mammary epithelial cells excretes milk through its apical surface into the apical compartment, thereby producing milk in culture.

32. The method of claim 31, wherein the basal compartment comprises a basal culture medium and the basal culture medium is in contact with the basal surface of the continuous polarized monolayer of primary mammary epithelial cells, with the basal surface of the continuous polarized the monolayer of the mixed population, or with the basal surface of the continuous polarized monolayer of live immortalized mammary epithelial cells.
33. The method of claim 31 or claim 32, wherein the culturing is carried out at a temperature of about 35° C. to about 39° C., optionally about 37° C.
34. The method of any one of claims 31 to 33, wherein the culturing is carried out at an atmospheric concentration of CO₂ of about 4% to about 6%, optionally about 5%.
35. The method of any one of claims 31 to 34, wherein the culturing comprises monitoring the concentration of dissolved O₂ and CO_{2.}
36. The method of claims 31 to 35, further comprising adding prolactin to the basal culture medium, thereby providing a lactogenic culture medium.
37. The method of any one of claims 31 to 36, wherein the culturing comprises monitoring the glucose concentration and/or rate of glucose consumption in the basal culture medium and/or in the lactogenic culture medium.
38. The method of claim 37, wherein the prolactin is added when the rate of glucose consumption is steady state.
39. The method of any one of claims 36 to 38, wherein the prolactin is produced by a microbial cell or a human cell expressing a recombinant prolactin (e.g., S179D-prolactin).
40. The method of any one of claims 31 to 39, further comprising collecting the milk from the apical compartment to produce collected milk.
41. The method of claim 40, wherein the collecting is via a port.
42. The method of claim 40 or claim 41, wherein the collecting is via gravity or a vacuum, optionally the vacuum is attached to the port.
43. The method of any one of claims 40 to 42, further comprising freezing the collected milk to produce frozen milk and/or lyophilizing the collected milk to produce lyophilized milk.
44. The method of any one of claims 40 to 43, further comprising packaging the collected milk, the frozen milk and/or the lyophilized milk into a container.
45. The method of any one of claims 40 to 42, further comprising extracting one or more components from the collected milk.
46. The method of claim 45, wherein the components from the collected milk are lyophilized or concentrated to produce a lyophilized or a concentrated milk component product.
47. The method of claim 46, wherein the components from the collected milk are concentrated by membrane filtration or reverse osmosis.
48. The method of any one of claims 45 to 47, wherein the lyophilized or concentrated milk component product is packaged in a container.
49. The method any one of claims 45 to 48, wherein the components from the collected milk are milk protein, lipid, carbohydrate, vitamin, and mineral contents.
50. The method of claim 48 or claim 49, wherein the container is sterile.
51. The method of any one of claims 48 to 50, wherein the container is vacuum-sealed
52. The method of any one of claims 48 to 51, wherein the container is a food grade container.
53. The method of any one of claims 48 to 52, wherein the container is a canister, a jar, a bottle, a bag, a box, or a pouch.
53. A method of producing a modified primary mammary epithelial cell or a immortalized mammary epithelial cell, wherein the method comprises introducing into the cell:

(a) a polynucleotide encoding a prolactin receptor comprising a modified intracellular signaling domain, optionally wherein the prolactin receptor comprises a truncation wherein position 154 of exon 10 has been spliced to the 3′ sequence of exon 11;

(b) a polynucleotide encoding a chimeric prolactin receptor that binds to a ligand, which is capable of activating milk synthesis in the absence of prolactin;

(c) a polynucleotide encoding a constitutively or conditionally active prolactin receptor protein, optionally wherein the polynucleotide encodes a constitutively active human prolactin receptor protein comprising a deletion of amino acids 9 through 187;

(d) a polynucleotide encoding a modified (recombinant) effector of a prolactin protein comprising (i) a JAK2 tyrosine kinase domain fused to a STATS tyrosine kinase domain; and/or (ii) a prolactin receptor intracellular domain fused to a JAK2 tyrosine kinase domain;

(e) a loss of function mutation into a circadian related gene PER2 (period circadian protein homolog 2); and/or

(f) a polynucleotide encoding one or more glucose transporter genes GLUT1 and/or GLUT12, thereby increasing the rate of nutrient uptake at the basal surface of the monolayer.

55. The method of claim 53, wherein the JAK2 tyrosine kinase domain is fused to the C-terminus of the STAT5 tyrosine kinase domain (e.g., a polynucleotide encoding a JAK2 tyrosine kinase domain is linked to the 3′ end of a polynucleotide encoding the STAT5 tyrosine kinase domain).
56. The method of claim 53 wherein the loss of function mutation comprises an 87-amino acid deletion from position 348 to 434 in PER2.

Having described the present invention, the same will be explained in greater detail in the following examples, which are included herein for illustration purposes only, and which are not intended to be limiting to the invention.

### EXAMPLES

### Example 1

A cell culture system designed for the collection of milk should support compartmentalized secretion of the product such that the milk is not exposed to the media that provides nutrients to the cells. In the body, milk-producing epithelial cells line the interior surface of the mammary gland as a continuous monolayer. The monolayer is oriented such that the basal surface is attached to an underlying basement membrane, while milk is secreted from the apical surface and stored in the luminal compartment of the gland, or alveolus, until it is removed during milking or feeding. Tight junctions along the lateral surfaces of the cells ensure a barrier between the underlying tissues and the milk located in the alveolar compartment. Therefore, in vivo, the tissue of the mammary gland is arranged such that milk secretion is compartmentalized, with the mammary epithelial cells themselves establishing the interface and maintaining the directional absorption of nutrients and secretion of milk.

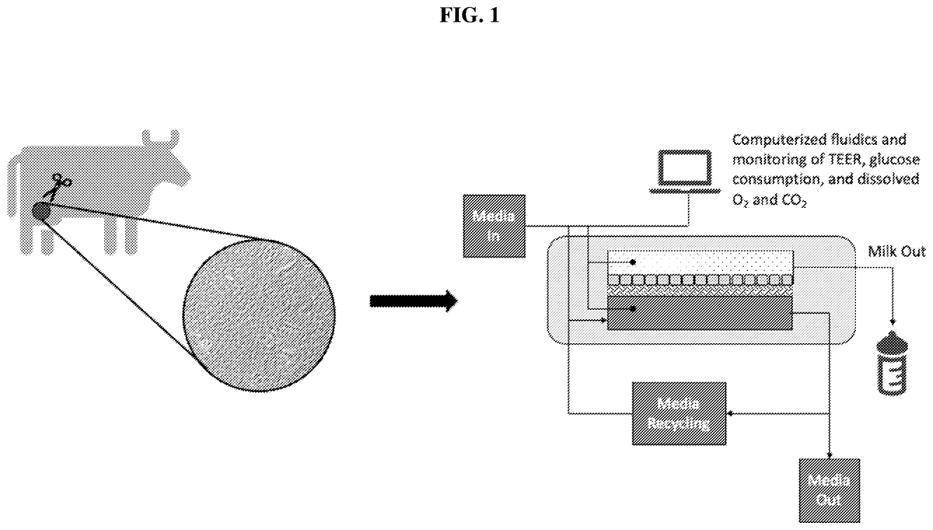

The present invention describes a cell culture apparatus that recapitulates the compartmentalizing capability of the mammary gland that may be used to collect milk from mammary epithelial cells grown outside of the body. Such an apparatus can include a scaffold to support the proliferation of mammary cells at the interface between two compartments, such that the epithelial monolayer provides a physical boundary between the nutrient medium and the secreted milk. In addition to providing a surface for growth, the scaffold provides spatial cues that guide the polarization of the cells and ensures the directionality of absorption and secretion. This invention describes the preparation, cultivation, and stimulation of mammary epithelial cells in a compartmentalizing cell culture apparatus for the production and collection of milk for nutritional use (see e.g., FIG. 1).

Preparation of mammary epithelial cells. Mammary epithelial cells are obtained from surgical explants of dissected mammary tissue (e.g., breast, udder, teat). Generally, after surgical dissection of the mammary tissue, any fatty or stromal tissue is manually removed under aseptic conditions, and the remaining tissue of the mammary gland is enzymatically digested with collagenase and/or hyaluronidase prepared in a chemically defined nutrient media, which should be composed of ingredients that are “generally recognized as safe” (GRAS). The sample is maintained at 37° C. with gentle agitation. After digestion, a suspension of single cells or organoids is collected, either by centrifugation or by pouring the sample through a sterile nylon cell strainer. The cell suspension is then transferred to a tissue culture plate coated with appropriate extracellular matrix components (e.g., collagen, laminin, fibronectin).

Alternatively, explant specimens can be processed into small pieces, for example by mincing with a sterile scalpel. The tissue pieces are plated onto a suitable surface such as a gelatin sponge or a plastic tissue culture plate coated with appropriate extracellular matrix.

The plated cells are maintained at 37° C. in a humidified incubator with an atmosphere of 5% CO₂. During incubation, the media is exchanged about every 1 to 3 days and the cells are sub-cultured until a sufficient viable cell number is achieved for subsequent processing, which may include preparation for storage in liquid nitrogen; development of immortalized cell lines through the stable transfection of genes such as SV40, TERT, or other genes associated with senescence; isolation of mammary epithelial, myoepithelial, and stem/progenitor cell types by, for example, fluorescence-activated cell sorting; and/or introduction into a compartmentalizing tissue culture apparatus for the production and collection of milk for human consumption.

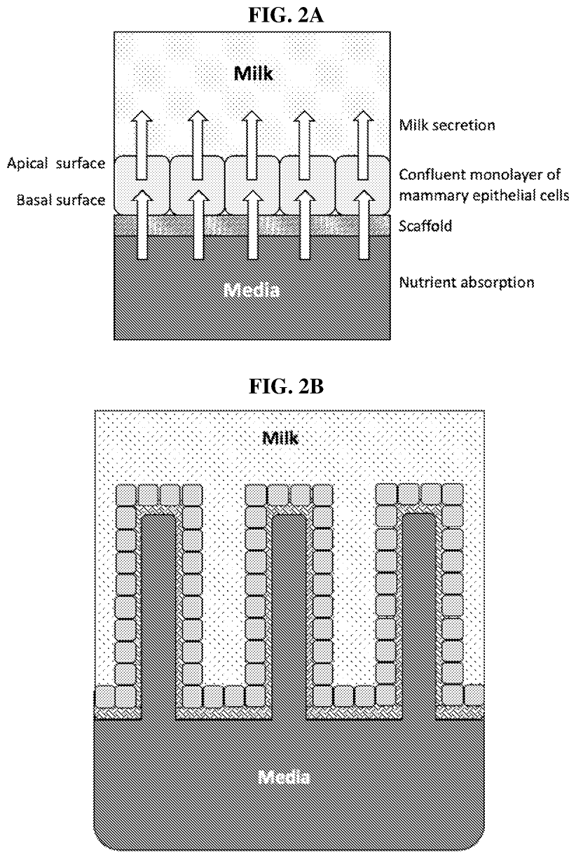

Cultivation of mammary epithelial cells for the production of milk. Milk for nutritional use is produced by mammary epithelial cells isolated as described above and cultured in a format that supports compartmentalized secretion such that separation between the nutrient medium and the product is maintained. The system relies on the ability of mammary epithelial cells to establish a continuous monolayer with appropriate apical-basal polarity when seeded onto an appropriate scaffold positioned at the interface between the apical compartment, into which milk is secreted, and the basal compartment, through which nutrient media is provided (see, e.g., FIG. 2). Transwell filters placed in tissue culture plates, as well as bioreactors based on hollow fiber or microstructured scaffolds, for example, may be used to support these characteristics.

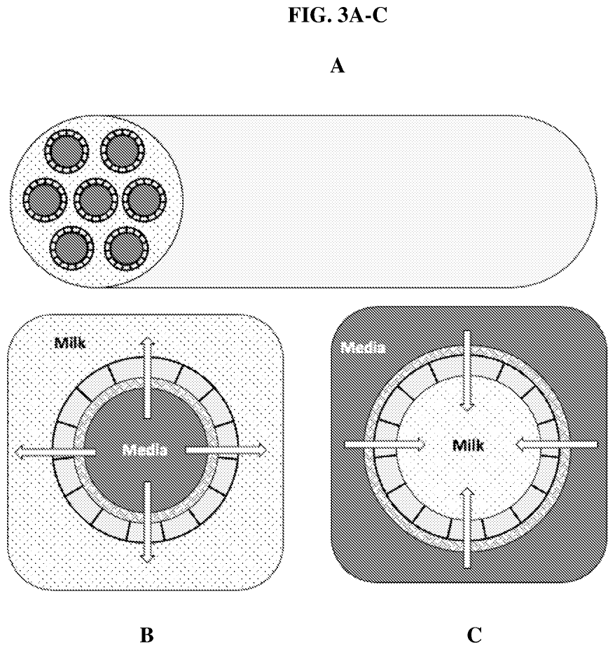

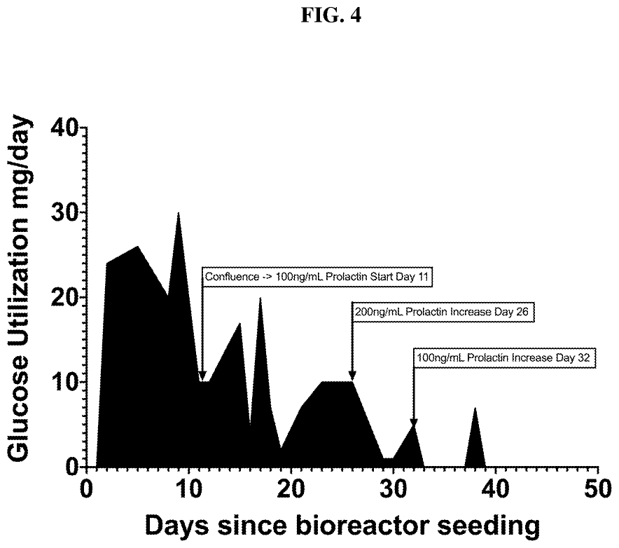

Following the isolation and expansion of mammary epithelial cells, the cells are suspended in a chemically defined nutrient medium composed of food-grade components and inoculated into a culture apparatus that has been pre-coated with a mixture of extracellular matrix proteins, such as collagen, laminin, and/or fibronectin. The cell culture apparatus may be any design that allows for the compartmentalized absorption of nutrients and secretion of product from a polarized, confluent, epithelial monolayer. Examples include hollow fiber and microstructured scaffold bioreactors (see, e.g., FIGS. 3 and 4, respectively). Alternatives include other methods of 3-dimensional tissue culture, such as the preparation of decellularized mammary gland as a scaffold, repopulated with stem cells to produce a functional organ in vitro, or collection of milk from the lumen of mammary epithelial cell organoids or “mammospheres” grown either in a hydrogel matrix or in suspension.

The apparatus includes sealed housing that maintains a temperature of about 37° C. in a humidified atmosphere of about 5% CO₂. Glucose uptake is monitored to evaluate the growth of the culture as the cells proliferate within the bioreactor. Stabilization of glucose consumption indicates that the cells have reached a confluent, contact-inhibited state. The integrity of the monolayer is ensured using transepithelial electrical resistance. Sensors monitor concentrations of dissolved O₂ and CO₂ in the media at multiple locations. A computerized pump circulates media through the bioreactor at a rate that balances the delivery of nutrients with the removal of metabolic waste such as ammonia and lactate. Media can be recycled through the system after removal of waste using Lactate Supplementation and Adaptation technology (Freund et al. 2018 Int J Mol Sci. 19(2)) or by passing through a chamber of packed zeolite.

Stimulation of milk production. In vivo and in cultured mammary epithelial cells, the production and secretion of milk is stimulated by prolactin. In culture, prolactin can be supplied exogenously in the nutrient media at concentrations approximating those observed in the body during lactation, e.g., about 20 ng/ml to about 200 ng/mL. Purified prolactin can be obtained commercially; however, alternative methods of providing prolactin or stimulating lactation may be employed, including expression and purification of the recombinant protein from microbial or mammalian cell cultures. Alternatively, conditioned media prepared by culturing cells that express and secrete prolactin can be applied to mammary epithelial cell cultures to stimulate lactation. Bioreactors can be set up in series such that media passing through a culture of cells expressing prolactin or other key media supplements is conditioned prior to exposure to mammary cells grown in a compartmentalizing culture apparatus as described.

Other approaches to upregulate milk production and/or spare the use of exogenous prolactin include molecular manipulation of the signaling pathways that are regulated by binding of prolactin to its receptor on the surface of mammary epithelial cells, such as the following: (a) expression of constructs targeting the posttranslational modification of prolactin; (b) expression of alternative isotypes of the prolactin receptor; (c) expression of a chimeric prolactin receptor in which the extracellular domain is exchanged with the binding site for a different ligand; (d) introduction of a gene encoding a constitutively or conditionally active prolactin receptor or modified versions of its downstream effectors such as STATS or Akt; (e) knockout or modification of the PER2 circadian gene; and/or (f) molecular approaches aimed at increasing the rate of nutrient uptake at the basal surface of the mammary epithelial monolayer.

Collection of milk. Secreted milk is collected continuously or at intervals through, for example, a port installed in the apical compartment of the culture apparatus. A vacuum may be applied to the port to facilitate collection and may also contribute to the stimulation of further production. The collected milk may be packaged into sterile containers and sealed for distribution, frozen or lyophilized for storage, or processed for the extraction of specific components.

The present invention provides mammary epithelial cell cultures for the production of milk for nutritional use. In addition to human breast milk, this method may be used to produce milk from other mammalian species, for example, for human consumption or veterinary use. Because it has not been previously possible to produce milk outside the body, this technology may result in novel commercial opportunities, in addition to providing an alternative mode of production for existing products. The social and economic effects of the commercial development of this technology are broad and far reaching. Production of human breast milk from cultured cells may provide a means to address infant malnutrition in food-scarce communities, provide essential nutrients to premature infants who are unable to breastfeed, and offer mothers a new option for feeding their babies that provides optimal nutrition with the convenience of infant formula. Production of cow or goat milk provides an opportunity to reduce the environmental, social, and animal welfare effects of animal agriculture. The process described here addresses an important gap in the emerging field of cellular agriculture and introduces an opportunity to dramatically update the human food supply without compromising our biological and cultural attachment to the most fundamental of our nutrition sources.

### Example 2

This example describes the successful production of a biosynthetic human milk product in a hollow fiber bioreactor seeded with primary human mammary epithelial cells (HMECs). As discussed in detail below, analyses of the biosynthetic milk product demonstrated that it contains many of the same compounds found in human milk, including many compounds not previously produced in a non-genetically engineered, fully human system.

The methods described here provide a proof-of-concept for the production of a non-genetically modified human biosynthetic milk product using a process that is readily scalable for commercial production. The hollow fiber bioreactor is a particularly advantageous system for maximizing surface area while allowing the cells to organize into three dimensional structures ideal for milk production and secretion. This cell culture systems allows the cells to achieve both the density and complexity needed to produce a full complement of milk molecules, including peptides, proteins, lipids, and carbohydrates, especially oligosaccharides. In the example below, a relatively small bioreactor cartridge (400 cm² surface area) produced about 30 milligrams (mg) of milk protein per day. As described in more detail below, this system can readily be adapted to a gram per day scale (e.g., 1-3 grams per day), for example by using larger commercially available bioreactor cartridges.

The process described here also utilizes food grade materials, including basement membrane and media components, in a pathogen free environment for culturing the lactating primary HUMECs. Thus, the resulting biosynthetic human milk product does not require pasteurization, unlike milk products made from extracts of bovine or human donor milk. It is well known that pasteurization reduces or destroys the immunological and nutritional bioactivity of many milk components, including important molecules such as bile salt-activated lipase (BSAL) and lysozyme. Accordingly, the human biosynthetic milk product described here is expected to have superior nutritional properties as well as other unique properties conferred by the provision of bioactive molecules, such as antimicrobial and anti-inflammatory molecules, as compared to pasteurized milk products.

The following paragraphs describe the culture of lactating monolayers of primary HUMECs in a small (400 cm² surface area) hollow fiber bioreactor and provide the initial characterization of the biosynthetic human milk secreted by these cells.

### Expansion of Primary Human Mammary Epithelial Cells (HMECs):

HMECs were obtained from the ATCC (PCS-600-010). HMECs (1 ampoule; 5×10⁵ cells) were expanded into a collagen-IV-coated T300 flask (or 2 T175 flasks) in mammary epithelial cell medium (ATCC PCS-600-30). Once an appropriate cell number was obtained, but prior to reaching confluence, the HMECs were detached, resuspended in growth medium, and seeded into the hollow fiber bioreactor, which was prepared as described below.

### Preparation of Hollow Fiber Bioreactor:

The cell culture apparatus used was a hollow fiber bioreactor that allows for the compartmentalized absorption of nutrients and secretion of milk product from a polarized, confluent, epithelial monolayer (se e.g., FIG. 3 and FIG. 4A-C). Such a bioreactor is made from capillaries fabricated from PVDF, polysulfone, or other biologically suitable materials assembled into a cylindrical cartridge. Cells are seeded into the extracapillary (EC) space and media is pumped through the capillaries, into the intra capillary space (IC). An illustrative schematic is shown in FIG. 4B.

Prior to seeding with cells, the cartridge was prepared by incubation with PBS for a minimum of 24 hours followed by coating the capillaries with a 1:1 mixture of collagen IV and laminin I (25 μg Laminin-111, 25 μg Collagen IV) in PBS at room temperature overnight. The collagen/laminin mixture was then exchanged with cell growth medium and incubated overnight at room temperature.

### Cell Growth in the Bioreactor:

After seeding, the HMECs were allowed to proliferate within the bioreactor for 10 days, based on the time needed to reach confluence as determined by glucose utilization. Glucose utilization is an indicator of cellular metabolism. During exponential growth, glucose utilization increases sharply, then slows and drops to a lower steady state when the cells reach confluence. As expected, and as shown in FIG. 4, glucose utilization increased rapidly for several days following seeding of the bioreactor, then leveled off and fell to a low stable level around day 10, indicating that the cells had reached confluence. When confluent, the monolayer formed a barrier dividing the intracapillary (IC) and extracapillary (ECS) spaces.

HMECs were cultured in the bioreactor using a basal mammary epithelial cell growth medium (ATCC® PCS-600-030™) supplemented with Dulbecco's Modified Eagle's Medium (DMEM, Sigma Aldrich) containing a chemically defined medium for high density cell culture (FiberCellSystems CDM-HD). The amount of DMEM/CDM-HD was adjusted based on the rate of glucose utilization. Once glucose utilization stabilized at under 10 mg/day (see FIG. 4) DMEM/CDM-HD was added to the basal growth medium in an amount of 10% by volume. This was to boost glucose content prior to prolactin stimulation in order to make the glucose more available as a carbon source for lactation.

In addition, once glucose utilization stabilized, indicating that the cells had reached confluence, a sample was extracted daily from the ECS (“ECS harvest”) and frozen for subsequent analyses of protein, lipid, and carbohydrate content as described in detail below. Samples were collected from the port hole of the ECS chamber using a syringe, centrifuged, and supernatants collected, divided into 0.5 mL aliquots, and frozen at −80° C. for analysis. Pelleted debris from the centrifugation step was resuspended in a volume of PBS equivalent to the original sample and frozen at −80° C. Milk production was stimulated by addition of prolactin to the media.

### Stimulation of Milk Production:

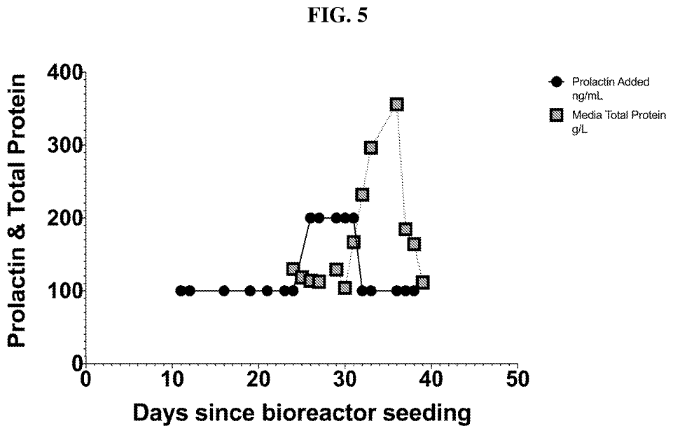

At day 11, after the initial stabilization of glucose utilization, media was supplemented with 100 ng/mL of prolactin to prime the cells for lactation (first arrow in FIG. 4). While 100 ng/mL is generally understood to be the serum concentration of prolactin in human mothers during lactation, it has been shown that at term prolactin levels are considerably higher, about 200 ng/mL. Therefore, we tested whether increasing the amount of prolactin after a period of time would be effective to stimulate milk production in the bioreactor cultivated cells. After 15 days of supplementation with 100 ng/ml prolactin, and at day 26 of culture in the bioreactor, the amount of prolactin was increased to 200 ng/ml (second arrow in FIG. 4). As shown in FIG. 5, the total protein production increased rapidly, by a factor of 4-5, within about 5 days of increasing prolactin to 200 ng/ml. In addition, the later decrease in prolactin concentration (third arrow in FIG. 4) correlated with a decrease in total protein production, indicating that total protein production can be controlled by varying the amount of prolactin. Without being bound by any theory, we believe that the increase in prolactin to 200 ng/ml following an initial exposure to 100 ng/ml prolactin is critical for activation of maximum lactation in the bioreactor cultivated cells.

### Characterization of Biosynthetic Milk Product

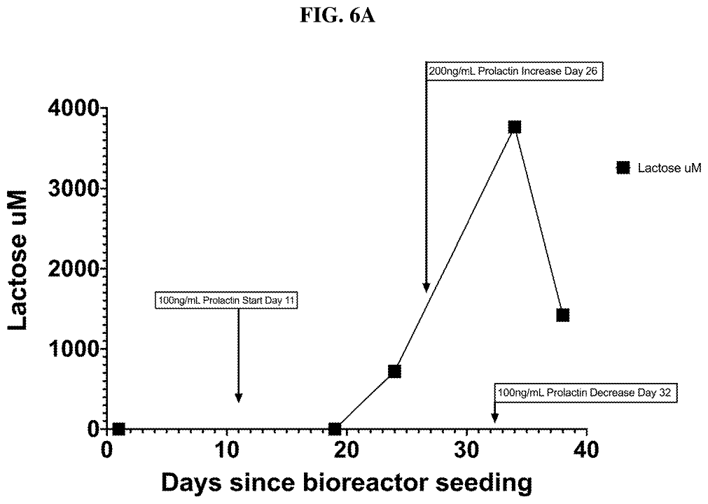

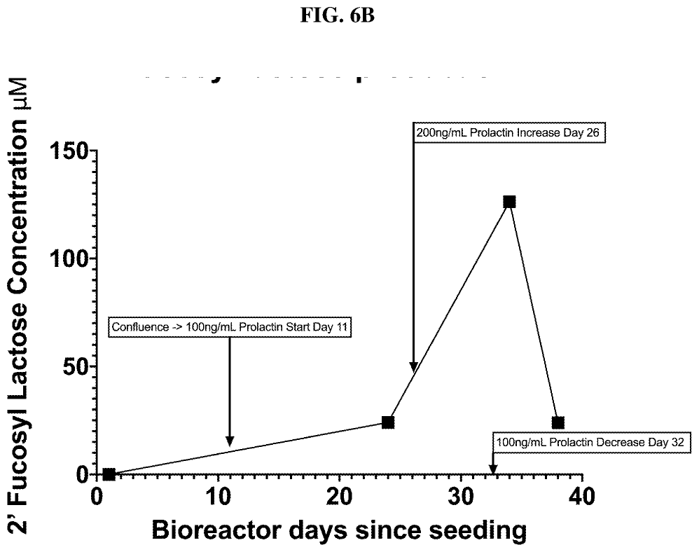

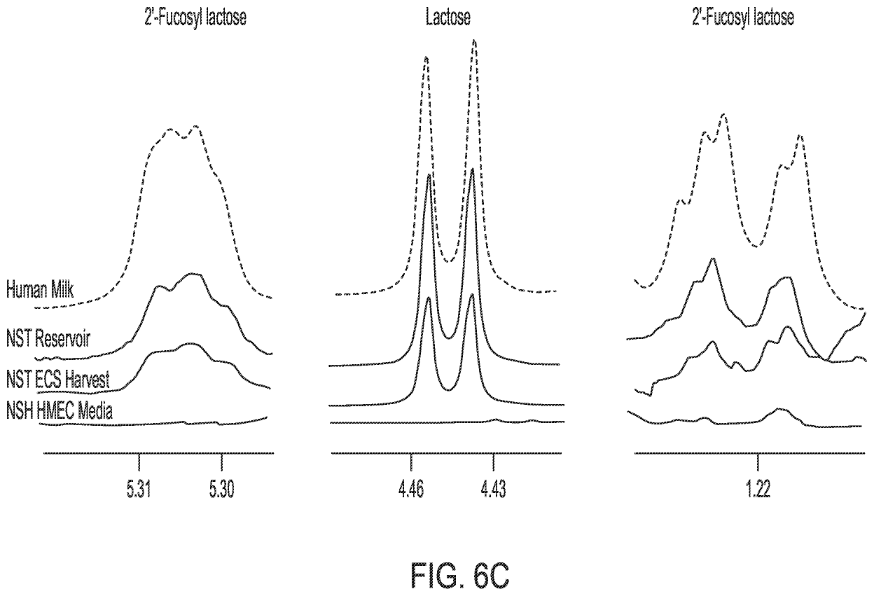

Lactose synthesis is the rate limiting step for milk production (Mahmoud et al. Am J Physiol Endocrinol Metab. 2012;303(3):E365-376.). In addition, lactose is also the primary carbohydrate in virtually all mammalian milks. Its presence is an indicator of successful mammalian milk biosynthesis. Accordingly, we analyzed the ECS harvests for lactose after prolactin stimulation. Lactose was detected using an enzymatic assay (Lactose Assay Kit, Sigma Aldrich). FIG. 6A shows lactose concentration (micromolar, uM) over time after seeding of the cells into the bioreactor. The figure shows that lactose production increased dramatically following the increase in prolactin to 200 ng/ml on day 26.

Human milk also contains functional non-nutritional components, including metabolites in the form of lipids, amino acids, biogenic amines and carbohydrates, particularly in the form of oligosaccharides. The human milk metabolome is generally defined as the set of low molecular weight molecules (less than 1500 Da) found in human milk. Accordingly, we further analyzed the metabolite component of the biosynthetic milk product by nuclear magnetic resonance (NMR) using Chenomx NMR Suite software as described in Smilowitz et al., J. Nutr. 143: 1709-1718, 2013. This technique has been validated for human milk and provides a quantitative measurement of carbohydrate, amino acid and organic acid content.

Metabolite analysis identified successful biosynthesis of key human milk metabolites including 2′ fucosyl lactose, as well as lactose and myo-inositol. Milk is also a significant source of myo-inositol and its presence further indicates successful comprehensive mammalian milk biosynthesis. Myo-inositol is often added to infant formulas to ensure against potential deficiency during early neonatal development. 2′ fucosyl lactose is an oligosaccharide and it is the most prevalent human milk oligosaccharide (HMO) naturally present in human breast milk, making up about 30% of all of HMOs found in human milk. Presence of 2′ fucosyl lactose in the supernatant indicates that the bioreactor cells successfully made human oligosaccharides. FIG. 6B shows 2′ fucosyl lactose concentration (micromolar, uM) over time after seeding of the cells into the bioreactor. The figure shows that 2′ fucosyl lactose production increased dramatically following the increase in prolactin to 200 ng/ml on day 26.

In order to confirm that the lactose and 2′ fucosyl lactose were being secreted by the cells, and not simply present in the culture media, we also analyzed samples of the culture media, ECS harvest, and reservoir for the presence of these carbohydrate molecules. As shown in FIG. 6C, there is no evidence of these molecules in the cell culture media, but representative peaks of both lactose and 2′ fucosyl lactose are clearly evident in both the ECS harvest and reservoir samples. The human milk spectra is included for qualitative comparison, it is not to scale with the others.

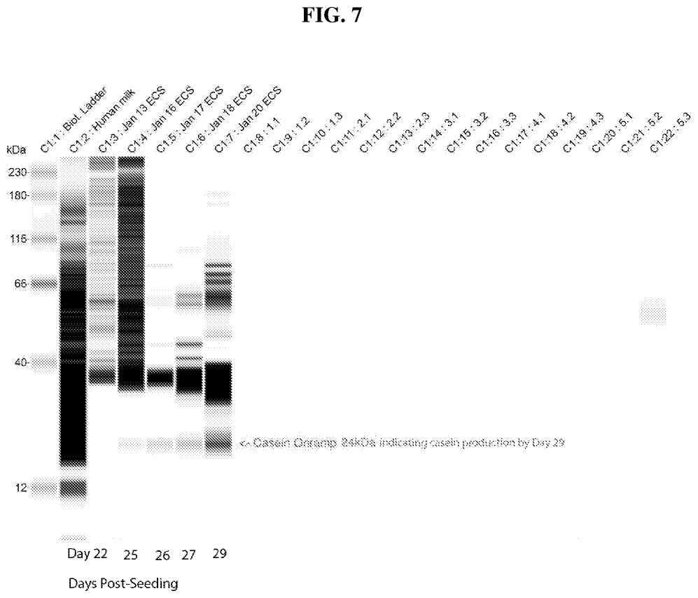

In addition to these important carbohydrates, we analyzed the biosynthetic milk product for human casein-2, which is one of the main proteins in human milk. FIG. 7 shows casein production over time using protein isolated from ECM harvest on days 22, 25, 26, 27, and 29 post-seeding of the bioreactor. Casein became detectable beginning at day 25 and continued to increase markedly over the next several days.

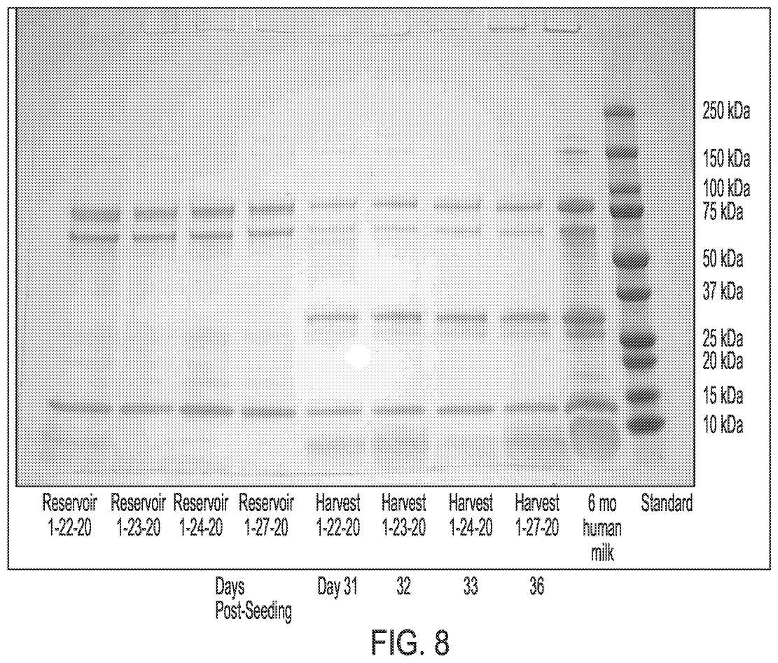

Analysis of the protein content of representative ECS harvests was performed using sodium dodecyl sulphate—polyacrylamide gel electrophoresis (SDS-PAGE) to visualize proteins having a molecular weight between 5 and 250 kDa. FIG. 8 shows an image of a Coomassie stained gel loaded with four reservoir samples (lanes 1-4 from the left) and four ECS harvest samples (lanes 5-8) corresponding to day 20, 21, 22, and 25 after prolactin stimulation. Lane 9, next to the molecular weight marker, shows protein from human milk for comparison. The reservoir samples were obtained from the intracapillary (IC) space of the bioreactor (the space internal to the capillaries, as shown in FIG. 3B), which contained the growth medium. These results indicated that the bioreactor produced a biosynthetic milk product containing many of the same proteins as are found in human milk.

The ECS harvest from lane 5 was further analyzed by liquid chromatography and mass spectrophotometry (LC-MS) to identify the proteins present. Details of the LC-MS analytic methods are provided below. This analysis identified a total of 81 proteins originating from 67 protein groups. These proteins included alpha, beta, and kappa-caseins and alpha-lactalbumin as well as serum albumin, lactotransferrin, xanthine dehydrogenase/oxidase, butyrophilin, insulin, perilipin-2, and osteopontin.

TABLE 1
Protein component of biosynthetic milk product

| Uniprot accession number | Name | Description |
|---|---|---|
| Q06278\|AOXA_HUMAN | Aldehyde oxidase | Nutritional, lactose |
| | | synthesis |
| P00709\|LALBA_HUMAN | Alpha-lactalbumin | Nutritional, calcium |
| | | transport |
| P47710\|CASA1_HUMAN | Alpha-S1-casein | Immunological, antigen |
| | | presentation |
| P61769\|B2MG_HUMAN | Beta-2-microglobulin | Nutritional, excellent amino |
| | | acid balance |
| P05814\|CASB_HUMAN | Beta-casein | Nutritional, lipid and |
| | | vitamin digestion |
| P19835\|CEL_HUMAN | Bile salt-activated lipase | Immunological, T cell |
| | | regulation |
| Q13410\|BT1A1_HUMAN | Butyrophilin subfamily 1 | Immunological regulation |
| | member A1 | |
| P10909\|CLUS_HUMAN | Clusterin | Nutritional, Protein folding, |
| | | chaperone |
| P11021\|BIP_HUMAN | Endoplasmic reticulum | Nutritional, Catalyzes |
| | chaperone BiP | biosynthesis of long-chain |
| | | fatty acids |
| P15311\|EZRI_HUMAN | Ezrin | Nutritional, Key role in |
| | | glycolysis and |
| | | gluconeogenesis, protein |
| | | scaffolding |
| P49327\|FAS_HUMAN | Fatty acid synthase | Nutritional, Glucose |
| | | regulation |
| P04075\|ALDOA_HUMAN | Fructose-bisphosphate | Nutritional, Glucose |
| | aldolase A | regulation |
| P01308\|INS_HUMAN | Insulin | Key nutritional milk protein |
| F8WCM5\|INSR2_HUMAN | Insulin isoform 2 | Nutritional, Immunological, |
| | | Important role in intestinal |
| | | epithelial homeostasis and |
| | | promotes mucosal healing |
| P07476\|INVO_HUMAN | Involucrin | Nutritional, Major iron- |
| | | binding and multifunctional |
| | | protein with antimicrobial |
| | | activity |
| P07498\|CASK_HUMAN | Kappa-casein | Nutritional, Key enzyme in |
| | | triglyceride metabolism |
| Q08431\|MFGM_HUMAN | Lactadherin | Immunological, Bacteriolytic |
| | | function, immunoagent |
| | | adjuvant |
| P02788\|TRFL_HUMAN | Lactotransferrin | Antimicrobial, role in |
| | | mammary cell |
| | | differentiation |
| P06858\|LIPL_HUMAN | Lipoprotein lipase | Immunological, essential in |
| | | pathway for type I |
| | | immunity, binds |
| | | hydroxyapatite |
| Q6ZMR3\|LDH6A_HUMAN | L-lactate dehydrogenase A- | Nutritional, Helps catalyze |
| | like 6A | oligopeptides |
| P61626\|LYSC_HUMAN | Lysozyme C | Nutritional, Maintenance of |
| | | adipose tissue |
| P26038\|MOES_HUMAN | Moesin | Nutritional, Protects against |
| | | oxidative stress |
| P15941\|MUC1_HUMAN | Mucin-1 | Nutritional, Promotes |
| | | healthy cell-cell |
| | | interendothelial junctions in |
| | | gastrointestinal lining |
| Q99102\|MUC4_HUMAN | Mucin-4 | Nutritional, Multifunction, |
| | | regulates osmotic pressure |
| | | and mineral transport |
| P10451\|OSTP_HUMAN | Osteopontin | Nutritional, Enemy in purine |
| | | degredation |
| P62937\|PPIA_HUMAN | Peptidyl-prolyl cis-trans | Nutritional, lactose |
| | isomerase A | synthesis |
| P23284\|PPIB_HUMAN | Peptidyl-prolyl cis-trans | Nutritional, calcium |
| | isomerase B | transport |
| Q99541\|PLIN2_HUMAN | Perilipin-2 | Immunological, antigen |
| | | presentation |
| P32119\|PRDX2_HUMAN | Peroxiredoxin-2 | Nutritional, excellent amino |
| | | acid balance |
| Q9NPG4\|PCD12_HUMAN | Protocadherin-12 | Nutritional, lipid and |
| | | vitamin digestion |
| P30613\|KPYR_HUMAN | Pyruvate kinase PKLR | Immunological, T cell |
| | | regulation |
| P02768\|ALBU_HUMAN | Serum albumin | Immunological regulation |
| P47989\|XDH_HUMAN | Xanthine dehydrogenase/ | Nutritional, Protein folding, |
| | oxidase | chaperone |

Several of the above molecules are found naturally only in human milk and are sensitive to degredation from heat or irradiation pasteurization. In particular, bile salt-activated lipase (BSAL) plays an essential role in lipid digestion including absorption of cholesterol and triacylglycerol. BSAL is not found in bovine milk nor produced by infants at birth. Recombinant BSAL failed phase III clinical trials, likely due loss of fragile post-translation modifications and/or improper protein folding, either of which could have resulted in a significant loss of bioactivity. Due to its vital role in lipid absorption, BSAL is utilized in human donor milk concentrated to boost the caloric absorption of extremely low birth weight preterm infants.

Lysozyme is another important immunological molecule sensitive to degradation and consequent loss of bioactivity. Attempts to produce this molecule recombinantantly have failed to reproduce the bioactivity of the native protein found in mother's milk.

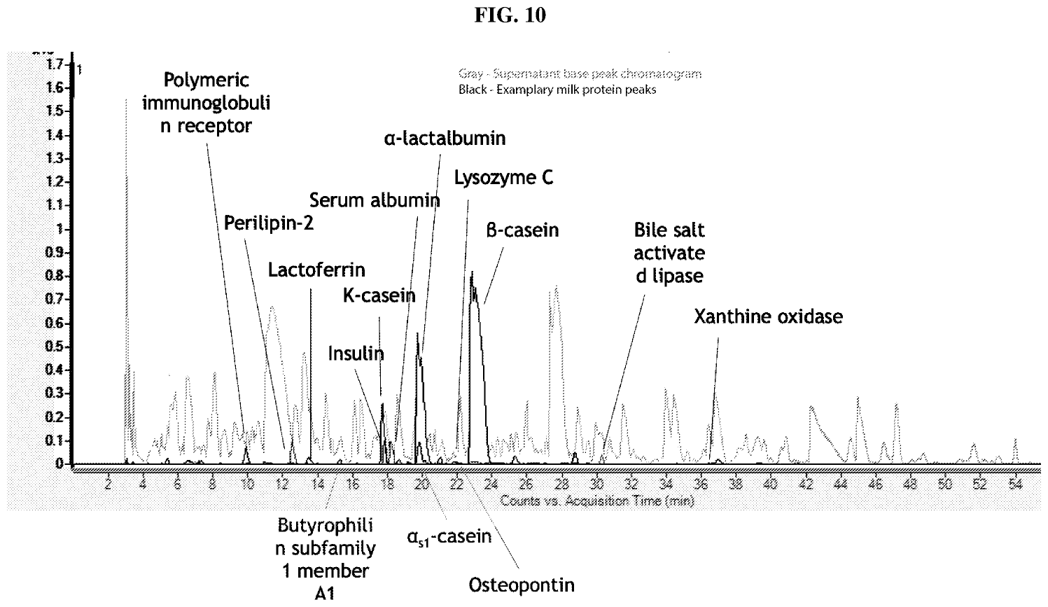

FIG. 10 shows the relative amounts of some key milk proteins in a sample of the biosynthetic milk product.

### General Methods for Sample Preparation and LC-MS Analysis

Proteins were digested and prepared for analysis by mass spectrometry essentially following “Basic Protocol 2,” steps 2-6, from Gundry, R. L. et al., Curr. Prot. Mol. Biol. 2009 10.25.1-10.25.23. The approximate protein content of each sample was determined with a Qubit Fluorometer (ThermoFisher Scientific, Waltham, Mass.).

The peptides were purified by microplate C18 solid phase extraction (Glygen Corp., Columbia, Md.). The solid phase was conditioned with 99.9% acetonitrile (ACN)/0.1% TFA and equilibrated with 1% ACN/0.1% TFA. The samples were loaded, and the solid phase was washed with 1.2 mL (approximately 6 column volumes) 1% ACN/0.1% TFA. The peptides were then eluted with 80% ACN/0.1% TFA and dried by vacuum centrifugation. The peptides were re-dissolved in 3% ACN for liquid chromatography-mass spectrometry (LC-MS) analysis.

Peptides were analyzed on an Agilent 6520 Accurate-Mass quadrupole time-of-flight (Q-TOF) LC-MS system. The nano-LC chip consisted of a 360 nL loading column and a 150 mm analytical column, both packed with C18. The analytical column was operated at a nanopump flow rate of 0.3 μL/min. The gradient elution solvents were (A) 3% ACN/0.1% FA and (B) 90% ACN/0.1% FA. Precursor ions were selected for tandem fragmentation if their intensity reached at least 1000 ion counts or 0.01% of the relative intensity of the spectra. Collision energies were specified based on the formula “Energy (V)=((m/z)/100)*slope+intercept,” with slope and intercept values of 3 and 2, respectively. Mass calibration was performed during data acquisition based on infused calibrant ions with m/z 322.048121 and 922.009798.

All spectra from each data file were saved as Agilent .d files and were analyzed by the proteomics software PEAKS Studio to identify peptides from the tandem-MS data. Carbamidomethylation of cysteine was set as a fixed modification. Oxidation of methionine, phosphorylation (serine, threonine, and tyrosine), deamidation (asparagine and glutamine), and carbamylation (lysine and N-terminus) were allowed as variable post-translational modifications. Precursor error tolerance was set to 20 ppm and ±0.035 Da was used for fragment ions. Maximum missed cleavages per peptide was set to 2. Peak integrations for label free quantification were conducted with a retention time window of 1 min and a mass error tolerance of 30 ppm. All peptide matches were identified at a 1% false discovery rate, and proteins were required to meet a −10log(P-value) threshold of at least 20.

### Analysis of Lipid Content

In addition to carbohydrates and proteins, lipids are an important component of mammalian milk. Oxylipins were extracted and identified by LC-MS as described below. Oxylipins are also referred to as bioactive lipid mediators of fatty acids. Table 2 below shows free oxylipin concentrations (nM) reported as average of the two independent ECS samples, along with the molecule's classification, if known. Comparative amounts identified by Gan et al. (Lipids 2020 November;55(6):661-670) in human skim milk are also shown for key molecules where the bioactive lipid was present in higher amounts in the ECS sample. The comparison with skim milk is appropriate because it captures dissloved lipids, which are the more biologically relevant lipids in milk. Comparing dissolved lipids in skim milk and bioreactor cultured cell supernatant provides a reasonable indicator of the quality of the lipid content of the biosynthetic milk product compared to milk. As evident from the lipids listed in the table below, many known anti-inflammatory lipids present in human milk were identified in the ECS samples. In addition, a number of lipids were present in the ECS samples at concentrations comparably higher than those reported by Gan et al.

TABLE 2
Lipid component of biosynthetic milk product

| | | | Conc. in skim |
|---|---|---|---|
| | | | milk per Gan et |
| Oxylipin | Conc. (nM) | Classification | al. |
| Resolvin E1 | 0.192 | Anti- | |
| | | inflammatory | |
| PGD3 | 0.0275 | | |
| 9,12,13-TriHOME | 50.3 | | |
| 9,10,13-TriHOME | 19.1 | | |
| PGE1 | 0.121 | | |
| PGD1 | 0.0104 | | |
| PGD2 | 0.056 | | |
| PGJ2 | 0.0787 | | |
| PGB2 | 0.405 | | |
| 8,15-DiHETE | 0.247 | | |
| 5,6-DiHETE | 0.450 | | |
| 5,15-DiHETE | 0.0624 | | |
| 17,18-DiHETE | 1.37 | | |
| 14,15-DiHETE | 1.95 | | ND |
| 11,12-DiHETE | 0.106 | | |
| 12,13-DiHOME | 1.56 | | |
| 9,10-DiHOME | 0.775 | | |
| 19,20-DiHPDA | 0.099 | | |
| 14,15-DiHETrE | 1.33 | | ND |
| 16,17-DiHPDA | 0.0310 | | |
| 11,12-DiHETrE | 0.368 | | ND |
| 9-HOTrE | 4.27 | Anti- | 2.25 |
| | | inflammatory | |
| 13-HOTrE | 3.06 | Anti- | 1.49 |
| | | inflammatory | |
| 8,9-DiHETrE | 0.0641 | | |
| 15-HEPE | 0.337 | Anti- | 0.33 |
| | | inflammatory | |
| 5,6-DiHETrE | 0.343 | | 0.15 |
| 8-HEPE | 0.112 | Anti- | ND |
| | | inflammatory | |
| 12-HEPE | 0.141 | Anti- | ND |
| | | inflammatory | |
| 5-HEPE | 0.444 | Anti- | 0.54 |
| | | inflammatory | |
| 13-HODE | 114 | | 4.5 |
| 9-HODE | 66.7 | | 6.89 |
| 15-HETE | 3.19 | | 0.1 |
| 17-HDoHE | 2.69 | Anti- | |
| | | inflammatory | |
| 13-oxo-ODE | 24.2 | | |
| 11-HETE | 1.87 | | |
| 15-oxo-ETE | 0.661 | Anti- | 0.02 |
| | | inflammatory | |
| 9-oxo-ODE | 23.8 | | |
| 8-HETE | 2.07 | | |
| 12-HETE | 1.88 | | |
| 9-HETE | 2.08 | | |
| 15(S)-HETrE | 1.35 | | |
| 12-oxo-ETE | 0.097 | Anti- | |
| | | inflammatory | |
| 5-HETE | 3.12 | | |
| 19(20)-EpDPE | 0.988 | Anti- | |
| | | inflammatory | |
| 12(13)-EpOME | 19.8 | | |
| 14(15)-EpETrE | 0.604 | | |
| 9(10)-EpOME | 11.7 | | |
| 13(14)-EpDPE | 0.163 | Anti- | |
| | | inflammatory | |
| 10(11)-EpDPE | 0.296 | Anti- | |
| | | inflammatory | |
| 5-oxo-ETE | 0.661 | Anti- | |
| | | inflammatory | |
| 11(12)-EpETrE | 0.405 | Anti- | |
| | | inflammatory | |
| 8(9)-EpETrE | 0.63 | Anti- | |
| | | inflammatory | |
| 5(6)-EpETrE | 0.677 | Anti- | |
| | | inflammatory | |

Abbreviations:
EpOME, epoxyoctadecenoic acid;
EpETrE, epoxyeicosatrienoic acid;
EpETE, epoxyeicosatetraenoic acid;
EpDPE, epoxydocosapentaenoic acid;
DiHOME, dihydroxyoctadecenoic acid;
DiHETrE, dihydroxyeicosatrienoic acid;
DiHETE, dihydroxyeicosatetraenoic acid;
HODE, hydroxyoctadecadienoic acid;
HETrE, hydroxyeicosatrienoicacid;
HETE, hydroxyeicosatetraenoic acid;
HOTrE, hydroxyoctadecatrienoic acid;
HEPE, hydroxyeicosapentaenoic acid;
HDoHE, hydroxydocosahexaenoic acid;
LT, leukotriene

### General Methods for Purification and Analysis of Bioactive Lipids

Unesterified lipids were extracted from two ECS samples weighing 33 and 74 mg, respectively. Samples were thawed on ice, spiked with 10 uL 2 uM of surrogate spike solution containing 9 deuterated surrogate standards and extracted in 600 uL methanol:water (1:4 v:v) containing 0.002% BHT, 250 uM EDTA and 0.01% acetic acid. The samples were vortexed for 5 sec. and centrifuged for 10 min at 13,000rpm, 0° C. The precipitated proteins were discarded and the remaining extract was subjected to solid phase extraction (SPE) using 100 mg tC18 Sep-Pak columns (Waters Corp). Oxylipins were eluted from the columns by gravity with 2 mL of methanol, dried under nitrogen and reconstituted with 100 uL LC-MS/MS grade methanol. Filtered oxylipin extracts were stored at −80 C until LC-MSMS analysis. All samples were analyzed within a week of oxylipin extraction using an Agilent 1290 Infinity UHPLC system coupled to an Agilent 6460 triple-quadrupole tandem mass spectrometer (Agilent, Santa Clara, Calif., USA) with electron spray ionization in negative mode. Analytes were captured using optimized dynamic Multiple Reaction Monitoring (dMRM) conditions following separation on a Zorbax Eclipse Plus C18 column (2.1×150 mm, 1.8 gm, Agilent, Santa Clara, Calif., USA, Cat # 959759-902). The auto-sampler and column were kept at 4 and 45 ° C., respectively. Mobile phase A was 0.1% acetic acid in Milli-Q water. Mobile phase B contained 0.1% acetic acid in acetonitrile/methanol (80:15, v/v).

### Production of Milk at Commercially Feasible Scale

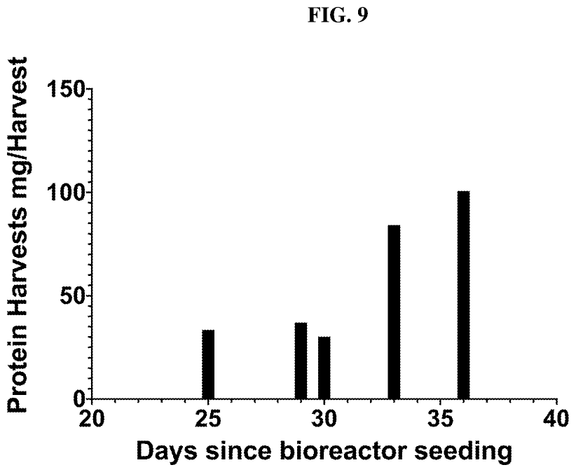

The methods described here provide a proof-of-concept for the production of a non-genetically modified human biosynthetic milk product using a process that is readily scalable for commercial production. As shown in FIG. 9, the amount of milk produced, analyzed as total secreted protein, was in the range of greater than 30 mg/day 5 days after increasing prolactin to 200 ng/mL. This daily production was sustained until the end of the experimental lactation period. This amount was obtained using a relatively small bioreactor cartridge (400 cm² surface area for cell growth). Using the largest commercially available bioreactor cartridge, which has a surface area of about 3 square meters (m²), this would translate into about 1-3 grams per day. The process is further scalable, for example, by packing more fibers and/or longer fibers into one or more cartridges aligned in parallel.

Together, the data presented here indicate that a substance similar to human milk was produced by the hollow fiber bioreactor cultivated HUMECs. Component analysis of the biosynthetic milk product produced by these cells demonstrates successful production of a full complement of human milk proteins, bioactive lipids, and carbohydrates, including key oligosaccharides. The biosynthetic milk product described here contained many important molecules not previously produced in a single product by other bioreactor based methods, some of which have proven difficult to manufacture by recombinant methods. These include lactose, bile salt-activated lipase, 2′ fucosyl lactose, lysozyme, and osteopontin. Further, the biosynthetic milk product produced here is pathogen free, without requiring pasteurization, and contains several antimicrobial human milk proteins such as lactoferrin and lysozyme. In addition, we have demonstrated here the feasibility of manufacturing a biosynthetic milk product at sufficient scale for use as a food product. To our knowledge, this represents the first method capable of producing human milk, or other mammalian milk, such as sheep, goat, or bovine, at a commercially feasible scale without requiring pasteurization. Since pasteurization is known to decrease or eliminate the activity of many proteins, including those that confer significant benefits to human milk, the process described here produces a milk product that is expected to have nutritional and other properties (e.g., antimicrobial) far superior to other forms of commercially produced milk.

The foregoing examples are illustrative of the present invention, and are not to be construed as limiting thereof. Although the invention has been described in detail with reference to preferred embodiments, variations and modifications exist within the scope and spirit of the invention as described and defined in the following claims.

What is claimed is:

1. A live cell construct comprising,
a scaffold having a top surface and a bottom surface; and
a continuous monolayer of (a) live primary mammary epithelial cells, (b) a mixed population of live primary mammary epithelial cells, mammary myoepithelial cells and mammary progenitor cells, and/or (c) live immortalized mammary epithelial cells on the top surface of the scaffold, the continuous monolayer of (a) live primary mammary epithelial cells, (b) mixed population of live primary mammary epithelial cells mammary myoepithelial cells and mammary progenitor cells, and/or (c) immortalized mammary epithelial cells having an apical surface and a basal surface (e.g., the cells form a polarized and confluent cell monolayer), wherein the construct comprises an apical compartment above and adjacent to the apical surface of the continuous monolayer of the (a) live primary mammary epithelial cells, the (b) mixed population of live primary mammary epithelial cells, mammary myoepithelial cell and mammary progenitor cells, and/or the (c) immortalized mammary epithelial cells and a basal compartment below and adjacent to the bottom surface of the scaffold.

2. The live cell construct of claim 1, wherein milk produced by the primary mammary epithelial cells or immortalized mammary epithelial cells is excreted through the apical surface of the cells into the apical compartment.

3. The live cell construct of claim 1 or claim 2, wherein the basal compartment comprises a basal culture medium and the basal culture medium is in contact with the basal surface of the live primary mammary epithelial cells, the mixed population of live primary mammary epithelial cells, mammary myoepithelial cells and mammary progenitor cells, and/or the immortalized mammary epithelial cells.

4. The live cell construct of claim 3, wherein the basal culture medium comprises a carbon source, a chemical buffering system, one or more essential amino acids, one or more vitamins and/or cofactors, and one or more inorganic salts.

5. The live cell construct of claim 3 or claim 4, wherein the basal culture medium is a lactogenic culture medium and further comprises prolactin.

6. The live cell construct of any one of claims 1 to 5, wherein the scaffold is fabricated as a 2-dimensional surface (e.g., a transwell filter), a 3-dimensional micropatterned surface (e.g., microstructured bioreactor, decellularized tissue), or as a cylindrical structure that can be assembled into bundles (e.g., hollow fiber bioreactor).

7. The live cell construct of any one of claims 1 to 6, wherein the top surface of the scaffold is coated with one or more extracellular matrix proteins.

8. The live cell construct of claim 6, wherein the one or more extracellular matrix proteins are collagen, laminin, entactin, tenascin, and/or fibronectin.

9. The live cell construct of any one of claims 1 to 8, wherein the scaffold comprises a natural polymer, a biocompatible synthetic polymer, a synthetic peptide, and/or a composite derived from any combination thereof.

10. The live cell construct of claim 9, wherein the natural polymer is collagen, chitosan, cellulose, agarose, alginate, gelatin, elastin, heparan sulfate, chondroitin sulfate, keratan sulfate, and/or hyaluronic acid.

11. The live cell construct of claim 9 or claim 10, wherein the biocompatible synthetic polymer may be polysulfone, polyvinylidene fluoride, polyethylene co-vinyl acetate, polyvinyl alcohol, sodium polyacrylate, an acrylate polymer, and/or polyethylene glycol.

12. The live cell construct of any one of claims 1 to 9, wherein said scaffold is porous.

13. The live cell construct of any one of claims 1 to 12, wherein the live primary mammary epithelial cells, the mixed population of live primary mammary epithelial cells, mammary myoepithelial cells and mammary progenitor cells, and/or the immortalized mammary epithelial cells are from a mammal.

14. The live cell construct of any one of claims 1 to 13, wherein the mammal is a primate (e.g., chimpanzee, orangutan, gorilla, monkey (e.g., Old World, New World), lemur, human), a dog, a cat, a rabbit, a mouse, a rat, a horse, a cow, a goat, a sheep, an ox, a pig, a deer, a musk deer, a bovid, a whale, a dolphin, a hippopotamus, an elephant, a rhinoceros, a giraffe, a zebra, a lion, a cheetah, a tiger, a panda, a red panda, and an otter.

15. The live cell construct of any one of claims 1 to 13, wherein the mammal is from an endangered species.

16. A method of producing milk in culture, the method comprising culturing the live cell construct of any one of claims 1 to 15, thereby producing milk in culture.

17. A method of making a live cell construct for producing milk in culture, the method comprising
(a) isolating primary mammary epithelial cells, myoepithelial cells and/or mammary progenitor cells from mammary explants from mammary tissue, to produce isolated mammary epithelial cells, myoepithelial cells and mammary progenitor cells;
(b) culturing the isolated primary mammary epithelial cells, myoepithelial cells and mammary progenitor cells to produce a mixed population of primary mammary epithelial cells, mammary myoepithelial cells and mammary progenitor cells;
(c) cultivating the mixed population of (b) on a scaffold, the scaffold having an upper surface and lower surface, to produce a polarized, continuous (i.e., confluent) monolayer of primary mammary epithelial cells, myoepithelial cells and mammary progenitor cells of the mixed population on the upper surface of the scaffold, wherein the polarized, continuous monolayer comprises an apical surface and a basal surface, thereby producing a live cell construct for producing milk in culture.

18. The method of claim 17, further comprising storing the isolated primary mammary epithelial cells, myoepithelial cells, and/or mammary progenitor cells of (b) prior to cultivating on a scaffold, optionally wherein the storing is in a freezer or in liquid nitrogen.

19. A method of making a live cell construct for producing milk in culture, the method comprising:
a) isolating primary mammary epithelial cells, myoepithelial cells, and/or mammary progenitor cells from mammary explants from mammary tissue (e.g., breast, udder, teat tissue), to produce isolated mammary epithelial cells, myoepithelial cells, and/or mammary progenitor cells;
(b) culturing the isolated primary mammary epithelial cells, myoepithelial cells, and/or mammary progenitor cells to produce a mixed population of primary mammary epithelial cells, mammary myoepithelial cells and mammary progenitor cells;
(c) sorting the mixed population of primary mammary epithelial cells, myoepithelial cells, and/or mammary progenitor cells to produce a population of primary mammary epithelial cells; and
(d) cultivating the population of primary mammary epithelial on a scaffold, the scaffold having an upper surface and lower surface, to produce a polarized, continuous (i.e., confluent) monolayer of primary mammary epithelial cells on the upper surface of the scaffold, wherein the polarized, continuous monolayer comprises an apical surface and a basal surface, thereby producing a live cell construct for producing milk in culture

20. A method of making a live cell construct for producing milk in culture, the method comprising
(a) culturing immortalized mammary epithelial cells to produce increased numbers of immortalized mammary epithelial cells;
(b) cultivating the immortalized mammary epithelial cells of (a) on a scaffold, the scaffold having an upper surface and lower surface, to produce a polarized, continuous (i.e., confluent) monolayer of immortalized mammary epithelial cells on the upper surface of the scaffold, wherein the polarized, continuous monolayer comprises an apical surface and a basal surface, thereby producing a live cell construct for producing milk in culture.

21. The method of claim 20, wherein prior to culturing immortalized mammary epithelial cells the method comprises:
(i) isolating primary mammary epithelial cells, myoepithelial cells, and/or mammary progenitor cells from mammary explants from mammary tissue (e.g., breast, udder, teat tissue), to produce isolated mammary epithelial cells, myoepithelial cells, and/or mammary progenitor cells;
(ii) culturing the isolated primary mammary epithelial cells, myoepithelial cells, and/or mammary progenitor cells to produce a mixed population of primary mammary epithelial cells, mammary myoepithelial cells and mammary progenitor cells;
(iii) sorting the mixed population of primary mammary epithelial cells, myoepithelial cells, and/or mammary progenitor cells to produce a population of primary mammary epithelial cells; and
(iv) stably transfecting one or more cells of the population of primary mammary epithelial cells of (iii) with one or more nucleic acids encoding hTERT or SV40; or transducing with a small hairpin RNA (shRNA) to p16 Inhibitor of Cyclin-Dependent Kinase 4) (p16(INK4)) and Master Regulator of Cell Cycle Entry and Proliferative Metabolism (c-MYC) to produce immortalized mammary epithelial cells.

22. The method of any one of claims 20, wherein the immortalized cell line is stably transfected with one or more nucleic acids encoding hTERT or SV40; or transduced with (a) a small hairpin RNA (shRNA) to p16 Inhibitor of Cyclin-Dependent Kinase 4) (p16(INK4)) and (b) Master Regulator of Cell Cycle Entry and Proliferative Metabolism (c-MYC).

23. The method of any one of claims 19 to 22, further comprising storing the population of primary mammary epithelial cells or the immortalized mammary epithelial cells prior to cultivating on a scaffold, optionally wherein the storing is in a freezer or in liquid nitrogen.

24. The method of any one of claims 17 to 23, wherein the basal surface of the monolayer is adjacent to the upper surface of the scaffold.

25. The method of any one of claims 17 to 24, wherein the live cell construct comprises an apical compartment that is adjacent to the apical surface of the monolayer.

26. The method of any one of claims 17 to 25, wherein the live cell construct comprises a basal compartment that is adjacent to the lower surface of the scaffold.

27. The method of any one of claims 17 to 26, wherein the culturing is carried out at a temperature of about 35° C. to about 39° C., optionally about 37° C.

28. The method of any one of claims 17 to 27, wherein the culturing is carried out at an atmospheric concentration of CO₂ of about 4% to about 6%, optionally about 5%.

29. The method of any one of claims 17 to 28, wherein the culturing of (b) comprises culturing in a culture medium that is exchanged about every day to about every 10 days, optionally about every day to about every 3 days.

30. The method of any one of claims 19 to 29, wherein the isolating and sorting is via fluorescence-activated cell sorting, magnetic-activated cell sorting, and/or microfluidic cell sorting.

31. A method of producing milk in culture comprising,
culturing a live cell construct comprising
(a) a scaffold comprising an upper surface and a lower surface and a continuous (i.e., confluent) polarized monolayer of live primary mammary epithelial cells, a continuous polarized monolayer of a mixed population of live primary mammary epithelial cells, mammary myoepithelial cells and mammary progenitor cells, and/or a continuous polarized monolayer of live immortalized mammary epithelial cells having an apical surface and a basal surface, wherein the continuous polarized monolayer of live primary mammary epithelial cells, the continuous polarized monolayer of the mixed population of live primary mammary epithelial cells, mammary myoepithelial cells and mammary progenitor cells and/or the continuous polarized monolayer of live immortalized mammary epithelial cells are located on the upper surface of scaffold,
(b) a basal compartment and an apical compartment, wherein the lower surface of the scaffold is adjacent to the basal compartment and the apical surface of the monolayer of live primary mammary epithelial cells, the monolayer of the mixed population of live primary mammary epithelial cells, mammary myoepithelial cells and mammary progenitor cells, and/or the monolayer of live immortalized mammary epithelial cells is adjacent to the apical compartment,
wherein the monolayer of live primary epithelial mammary cells, the live primary epithelial mammary cells of the monolayer of the mixed population of live primary mammary epithelial cells, mammary myoepithelial cells and mammary progenitor cells, or the monolayer of immortalized mammary epithelial cells excretes milk through its apical surface into the apical compartment, thereby producing milk in culture.

32. The method of claim 31, wherein the basal compartment comprises a basal culture medium and the basal culture medium is in contact with the basal surface of the continuous polarized monolayer of primary mammary epithelial cells, with the basal surface of the continuous polarized the monolayer of the mixed population, or with the basal surface of the continuous polarized monolayer of live immortalized mammary epithelial cells.

33. The method of claim 31 or claim 32, wherein the culturing is carried out at a temperature of about 35° C. to about 39° C., optionally about 37° C.

34. The method of any one of claims 31 to 33, wherein the culturing is carried out at an atmospheric concentration of CO₂ of about 4% to about 6%, optionally about 5%.

35. The method of any one of claims 31 to 34, wherein the culturing comprises monitoring the concentration of dissolved O₂ and CO₂.

36. The method of claims 31 to 35, further comprising adding prolactin to the basal culture medium, thereby providing a lactogenic culture medium.

37. The method of any one of claims 31 to 36, wherein the culturing comprises monitoring the glucose concentration and/or rate of glucose consumption in the basal culture medium and/or in the lactogenic culture medium.

38. The method of claim 37, wherein the prolactin is added when the rate of glucose consumption is steady state.

39. The method of any one of claims 36 to 38, wherein the prolactin is produced by a microbial cell or a human cell expressing a recombinant prolactin (e.g., S179D-prolactin).

40. The method of any one of claims 31 to 39, further comprising collecting the milk from the apical compartment to produce collected milk.

41. The method of claim 40, wherein the collecting is via a port.

42. The method of claim 40 or claim 41, wherein the collecting is via gravity or a vacuum, optionally the vacuum is attached to the port.

43. The method of any one of claims 40 to 42, further comprising freezing the collected milk to produce frozen milk and/or lyophilizing the collected milk to produce lyophilized milk.

44. The method of any one of claims 40 to 43, further comprising packaging the collected milk, the frozen milk and/or the lyophilized milk into a container.

45. The method of any one of claims 40 to 42, further comprising extracting one or more components from the collected milk.

46. The method of claim 45, wherein the components from the collected milk are lyophilized or concentrated to produce a lyophilized or a concentrated milk component product.

47. The method of claim 46, wherein the components from the collected milk are concentrated by membrane filtration or reverse osmosis.

48. The method of any one of claims 45 to 47, wherein the lyophilized or concentrated milk component product is packaged in a container.

49. The method any one of claims 45 to 48, wherein the components from the collected milk are milk protein, lipid, carbohydrate, vitamin, and mineral contents.

50. The method of claim 48 or claim 49, wherein the container is sterile.

51. The method of any one of claims 48 to 50, wherein the container is vacuum-sealed

52. The method of any one of claims 48 to 51, wherein the container is a food grade container.

53. The method of any one of claims 48 to 52, wherein the container is a canister, a jar, a bottle, a bag, a box, or a pouch.

54. A method of producing a modified primary mammary epithelial cell or an immortalized mammary epithelial cell, wherein the method comprises introducing into the cell:
(a) a polynucleotide encoding a prolactin receptor comprising a modified intracellular signaling domain, optionally wherein the prolactin receptor comprises a truncation wherein position 154 of exon 10 has been spliced to the 3′ sequence of exon 11;
(b) a polynucleotide encoding a chimeric prolactin receptor that binds to a ligand, which is capable of activating milk synthesis in the absence of prolactin;
(c) a polynucleotide encoding a constitutively or conditionally active prolactin receptor protein, optionally wherein the polynucleotide encodes a constitutively active human prolactin receptor protein comprising a deletion of amino acids 9 through 187;
(d) a polynucleotide encoding a modified (recombinant) effector of a prolactin protein comprising (i) a JAK2 tyrosine kinase domain fused to a STATS tyrosine kinase domain; and/or (ii) a prolactin receptor intracellular domain fused to a JAK2 tyrosine kinase domain;
(e) a loss of function mutation into a circadian related gene PER2 (period circadian protein homolog 2); and/or
(f) a polynucleotide encoding one or more glucose transporter genes GLUT1 and/or GLUT12, thereby increasing the rate of nutrient uptake at the basal surface of the monolayer.

55. The method of claim 54, wherein the JAK2 tyrosine kinase domain is fused to the C-terminus of the STATS tyrosine kinase domain (e.g., a polynucleotide encoding a JAK2 tyrosine kinase domain is linked to the 3′ end of a polynucleotide encoding the STATS tyrosine kinase domain).

56. The method of claim 54 wherein the loss of function mutation comprises an 87-amino acid deletion from position 348 to 434 in PER2.

57. A composition comprising a biosynthetic milk product produced by a live cell construct according to any one of claims 1-15.

58. A composition comprising a biosynthetic milk product produced by the method of any one of claims 16-18 or claims 31-54.

59. A live cell construct comprising lactating primary human mammary epithelial cells (HMECs) forming a continuous monolayer on a plurality of hollow capillary tubes arranged in a parallel array within a tubular cartridge defining an intracapillary (IC) space and an extracapillary (EC) space,
each hollow capillary tube constructed of a semi-permeable membrane defining an internal surface adjacent to the IC space and an external surface adjacent to the EC space,
wherein the external surface of each hollow capillary tube is coated with a mixture of collagen IV and laminin I and the HUMEC monolayer is in contact with the coated surface;
and wherein a cell growth medium supplemented with prolactin fills the IC space.

60. The live cell construct of claim 59, wherein the semi-permeable membrane is fabricated from polyvinylidene difluoride (PVDF) or polysulfone.

61. The live cell construct of claim 59 or 60, wherein the semi-permeable membrane has a molecular weight cut-off (MWCO) between 5-80 kilodaltons (kDa).

62. A composition comprising a biosynthetic human milk product produced by the live cell construct of any one of claims 59-61.

63. A biosynthetic human milk composition comprising a lipid component, a protein component, and a carbohydrate component, wherein the lipid, protein, and carbohydrate components each consist of human lipids, human proteins or peptides, and human carbohydrates, and wherein the composition is free of pathogens, cytotoxins, and genetically modified or engineered molecules.

64. The composition of claim 63, wherein the composition is not pasteurized.

65. The composition of claim 63 or 64, wherein the lipid component comprises 1-5% of the composition.

66. The composition of any one of claims 63-65, wherein the protein component comprises 0.5-1% of the composition.

67. The composition of any one of claims 63-66, wherein the carbohydrate component comprises 6-8% of the composition.

68. The composition of any one of claims 63-67, wherein the lipid component comprises palmitic acid, oleic acid, and one or more bioactive lipid mediators of fatty acids.

69. The composition of claim 68, wherein the one or more bioactive lipid mediators of fatty acids is an anti-inflammatory compound.

70. The composition of claim 68, wherein the one or more bioactive lipid mediators of fatty acids is selected from the group consisting of epoxyoctadecenoic acid (EpOME); epoxyeicosatrienoic acid (EpETrE); epoxyeicosatetraenoic acid (EpETE); epoxydocosapentaenoic acid (EpDPE); dihydroxyoctadecenoic acid (DiHOME); dihydroxyeicosatrienoic acid (DiHETrE); dihydroxyeicosatetraenoic acid (DiHETE); hydroxyoctadecadienoic acid (HODE); hydroxyeicosatrienoicacid (HETrE); hydroxyeicosatetraenoic acid (HETE); hydroxyoctadecatrienoic acid (HOTrE); hydroxyeicosapentaenoic acid (HEPE); hydro-xydocosahexaenoic acid (HdoHE); and leukotriene.

71. The composition of any one of claims 63-70, wherein the protein component comprises one or more proteins or peptides selected from the group consisting of alpha-lactalbumin, bile salt-activated lipase (BSAL), butyrophilin, casein, fatty acid synthase, insulin, lactadherin, lactoferrin, lactotransferrin, lysozyme, mucin-1, osteopontin, perilipin-2, serum albumin, and xanthine dehydrogenase/oxidase.

72. The composition of claim 71, wherein the protein component comprises BSAL, lysozyme, and lactoferrin.

73. The composition of any one of claims 63-72, wherein the carbohydrate component comprises one or more of lactose, 2′ fucosyl lactose, myo-inositol, lacto-N-neotetraose (LNnT), 6′-sialyllactose, sialyl-lacto-N-tetraose, lacto-N-fucopentaose (LNFP) I, lacto-N-fucopentaose (LNFP) II, and disialyl-lacto-N-tetraose.

74. The composition of any one of claims 63-73, wherein the composition is produced by the live cell construct of any one of claims 59-61.

75. A method for making a biosynthetic milk product, the method comprising expanding a population of human mammary epithelial cells (HUMECs) in a growth medium on a substrate comprising collagen IV; dislodging the expanded population of HUMECs from the substrate and seeding the dislodged HUMECs into a hollow fiber bioreactor containing capillaries pre-coated with a mixture of collagen IV and laminin I; culturing the HUMECs for a period of time until the HUMECs have reached confluence; and stimulating production of the biosynthetic milk product by contacting the HUMECs with prolactin using a method comprising contacting the cells with 100 ng/ml prolactin for a period of time followed by contacting the cells with 200 ng/ml prolactin for a second period of time.

76. The method of claim 75, wherein the HUMECs are selected from primary cells, primary immortalized cells, or recombinant cells.

77. The method of claim 75 or 76, further comprising a step of preparing the bioreactor prior to seeding the HUMECs, wherein preparing the bioreactor comprises creating a negative pressure within the bioreactor and applying a 1:1 mixture of collagen IV and laminin I in phosphate buffered saline (PBS) to the hollow fibers.

78. The method of claim 77, wherein applying the mixture of collagen IV and laminin I is accomplished using a syringe inserted into a port of the bioreactor.
